(19) United States
(12) Patent Application Publication
(10) Pub. No.: US 2022/0008438 A1
(43) Pub. Date: Jan. 13, 2022

(54) TMA-PHT Complex (Olive Triterpenic and Polyphenolic Compounds) and Their Combinations, As A Group of Phytochemical Drug Compositions and/or Pharmaceutical Products for The Treatment of COVID-19 and Other Human and Animal Diseases and Conditions

(71) Applicants: Alberto Sarmentero, Arlington, VA (US); Jose Prados Osuna, Granada (ES)

(72) Inventors: Alberto Sarmentero, Arlington, VA (US); Jose Prados Osuna, Granada (ES)

(21) Appl. No.: 17/370,768

(22) Filed: Jul. 8, 2021

Related U.S. Application Data
(60) Provisional application No. 63/050,065, filed on Jul. 9, 2020.

(51) Int. Cl. A61K 31/56; A61K 31/05; A61K 45/06; A61P 31/14

(52) U.S. Cl. CPC A61K 31/56; A61P 31/14; A61K 45/06; A61K 31/05

(57) ABSTRACT

TMA-PHT complex (olive triterpenic and polyphenolic compounds) and their combinations are used for preventing, alleviating, treating, or curing abnormal and/or pathological conditions of the living body, by such means as: (a) protecting the good health conditions of a living body by attracting, disabling, inhibiting, killing, modifying, repelling and/or retarding a virus or micro-organism in the case of preventing an infection (b) destroying a virus or a parasitic organism; (c) limiting the affecting of the disease by altering the physiology of the virus or parasite. In accordance with various embodiments, the present subject matter pertains to pharmaceutical drug compositions targeting various therapeutic objectives and their methods for preventing or treating Covid19 and/or other diseases and certain health conditions including the ones associated with inflammation and/or oxidative stress.

### CROSS-REFERENCE TO RELATED APPLICATIONS

This application claims priority to U.S. Provisional 63/050,065, filed Jul. 9, 2020, the entire disclosure of which is hereby incorporated herein by reference.

### TECHNICAL FIELD

The present invention relates, generally, to drug compositions containing several bioactive elements for preventing or treating certain health conditions, and, more particularly, to their methods of extraction, manufacturing, and usage.

### BACKGROUND

Viruses are the most abundant biological entities on the planet. These tiny acellular infectious agents—made up of genes that contain nucleic acids surrounded by proteins—are themselves also a key important natural mean of transferring genes between different organisms and played a central role in the evolution of all forms of life on Earth since Precambrian times. All viruses are obligate “parasites” that depend on the cellular machinery of their hosts to reproduce. Viruses are not active outside their hosts, which has led many to suggest that they are not exactly living organisms. Every living organism, including animals, plants, fungi, and bacteria, are virus hosts, but most infecting viruses have only one type of host.

In order to reproduce, viruses hijack the host cell factory to produce more viruses instead of new cells. RNA viruses carry their own genetic information copying machines (e.g., RNA polymerase enzyme) or possess genes (genetic information) that produce the proteins that are required to assemble the copying machines within the cell. They infect, making them independent of the cellular machinery and capable of infecting cells that are not actively reproducing.

SARS-CoV-2 viral infection determined the COVID-19 syndrome characterized, in the worst cases, by severe respiratory distress, pulmonary and cardiac fibrosis, release of inflammatory cytokines, and immunosuppression. This condition has caused the death of approximately 2.15% of the total world population infected so far. The highly contagious nature of COVID-19 dual disease along with the lack of any fully effective treatment —besides the several existing preventive vaccines, has implied extremely serious disruption and death around the world.

This zoonotic RNA virus SARS-CoV-2 responsible for the present-day pandemic, consists of an extremely small ball about 120 nanometers in diameter, almost as small for a human size as the same human is for the entire planet Earth. It also has a short genome written with combinations of our same four-letter molecular code (a, u, g, c) with which is written the RNA text that comprises several molecular keys ready to open various receptors of our cells that has caused the death of so many people since its existence in humans was first detected in late 2019.

The Covid-19 pandemic is one of the hardest challenges contemporary medicine and bioscience has ever faced. For this reason, effective treatments to prevent and/or cure this phasic disease are currently being sought around the world. In order to neutralize this virus through the appropriate use of antivirals, it must be considered not only the susceptibility to the infectious agent, but also the complex interaction that occurs between the antiviral, the patient and the virus: the pharmacokinetics and pharmacodynamics capable of overcoming COVID-19, a complex disease with a high potential for phasic progress from an initial syndrome typified by fever and respiratory symptoms, to one or more potential serious complications, including acute respiratory distress syndrome (ARDS), marked coagulopathy, hyper-inflammation and multi-organ failure.

Virtually all viral types have been found to infect plants, including single- and double-stranded RNA viruses as well as DNA viruses with and without lipid envelopes. That led some plants along their evolution to develop specific substances with strong antiviral properties to survive its infections. One of the most advanced plant in such virus-fighting experience along geological times is the Mediterranean Olive tree that produces a unique collection of several natural terpenes including some exclusive pentacyclic Triterpenes as well as a wider collection of 160 different polyphenols, both showing outstanding anti-viral properties as a result of its long history of fighting virus vulnerabilities in several instances of the viral attack (i.e. blocking proteins, spike, entrance, reproduction, etc.), while the good news is that some of these phytochemical compounds can be made quite amenable for human consumption to result very well bio-absorbed by targeted human cells if properly prepared, as we propose in this application.

A crucial problem in trying to take advantage of some of these antiviral vegetable substances was that pharmacologists were unable to transfer their effect to humans since the appropriate technologies had not been achieved until now to make them bioavailable for absorption and use by human cells. A proprietary original composition of several key phytochemical molecules —mainly obtained selectively from long-lived Olive trees has proven to behave as a safe drug mix against Covid19. This set of original molecules collectively grouped under the name OLIVEVIR™ and obtained mainly from long-lived olive trees, have been shown to be highly safe and effective drugs against various types of viral infections, including 7 therapeutic targets of Covid19 syndrome. This antiviral drug composition includes 20 TMA phytochemicals extracted from the Olive Triterpenic fraction and another 160 PHT phytochemicals from its Polyphenolic fraction. All of them are presented for the first time in a highly bioavailable complex for their effective absorption by human cells.

The following references are indicative of the levels of those skilled in the art to which the invention pertains. These documents and publications are incorporated herein by reference to the same extent as if each individual document or publication was specifically and individually incorporated herein by reference.
Kawase M, Shirato K, van der Hoek L, Taguchi F, Matsuyama S (June 2012). “Simultaneous treatment of human bronchial epithelial cells with serine and cysteine protease inhibitors prevents severe acute respiratory syndrome coronavirus entry”. J. Virol. 86 (12): 6537-45. doi:10.1128/JVI.00094-12. PMC 3393535. PMID 22496216.
Hoffman, Markus (May 2020). “SARS-CoV-2 Cell Entry Depends on ACE2 and TMPRSS2 and Is Blocked by a Clinically Proven Protease Inhibitor”. Cell. Retrieved 2020 Mar. 5.
Jose Prados Osuna, José Garcia-Granados Lopez de Hierro, Andrés; Parra Sánchez, Andrés y Martinez Rodriguez, Antonio. (November 2009), Uso del acido maslinico para el tratamiento de patologias y sus sintomas mediante la inhibición de COX-2. Pat No ES 2 328 999 A1
THE MEDICAL NEWS, (Feb. 14 2008) Maslinic acid from olives shows anti-cancer properties.
Cancer Lett. (January 2009) Maslinic acid from olives shows anti-cancer properties, Maslinic acid, a natural triterpene from Olea europaea L., induces apoptosis in HT29 human colon-cancer cells via the mitochondrial apoptotic pathway. 8; 273(1):44-54. Epub 2008 Sep. 14.
Gergerich, R. C., and V. V. Dolja. (2006) Introducción a los Virus Vegetales, el Enemigo Invisible. Translation. Silvina L. Giammaria. 2008 The Plant Health Instructor. DOI: 10.1094/PHI-I-2008-0122-01, 2006 https://www.apsnet.org/edcenter/disandpath/viral/introduction/Pages/PlantVirusesEspanol.aspx/
Kentaro Yamada et al. (2009) Mechanism of the antiviral effect of hydroxytyrosol on influenza virus appears to involve morphological change of the virus. Pub. AVR-2468; Elsevier.
Rafael de la Torre et al. (2020) Pharmacokinetics of maslinic and oleanolic acids from olive oil—Effects on endothelial function in healthy adults. A randomized, controlled, dose-response study. ELSEVIER. Food Chemistry Vol. 322, Aug. 30. 2020 https://www.sciencedirect.com/science/article/abs/pii/S0308814620305380?via%3Dihub.
Brooks M. Hybertson, et al, (May 2019), Phytochemical Combination PB125 Activates the Nrf2 Pathway and Induces Cellular Protection against Oxidative Injury. ResearchGate.https://www.researchgate.net/publication/332905722_.
Joe M. McCord, Brooks M. Hybertson, Adela Cota-Gomez, Bifeng Gao, (May 2020.) Nrf2 Activator PB125® as a Potential Therapeutic Agent Against COVID-19doi: https://doi.org/10.1101/2020.05.16.099788. Preprint BIORXIB.
Elise L. Donovan et al, (March 2012) Phytochemical Activation of Nrf2 Protects Human Coronary Artery Endothelial Cells against an Oxidative Challenge. Hindawi Publishing Corporation Oxidative Medicine Volume 2012, Article ID 132931.
Jesus Villar, M D et al. (March 2020) Dexamethasone treatment for the acute respiratory distress syndrome: A multicenter, randomized controlled trial. The Lancet Respiratory Medicine, Volume 8, Issue 3, Pages 267-276. https://www.sciencedirect.com/science/article/abs/pii/S2213260019304175
Lupiáñez, J. A., Garcia-Granados, A., Parra, A., Fernandez-Navarro, M., de la Higuera, M. y Peragón, J. (2005). Use of maslinic acid as a food additive. Application No.: PCT/ES2005000369. International Patent No.: WO 2006/003226 A1. Publication Date: 12 Jan. 6. Entity headline: University of Granada and University of Jaen. Countries to which it has spread: Everyone. Company that is exploding: BIOMASLINIC S.L./Now BLUE SINGULARITY SL.
Maria Luisa Hernández Jiménez (2008) Análisis metabólico y molecular del contenido de ácido linoleico en el fruto del olivo (Olea europea). José M. Martinez Rivas (director of the thesis), Universidad de Sevilla Spain dialnet.unirioja.es/servlet/tesis?codigo=150630.
B. Coutarda et al (April 2020) The spike glycoprotein of the new coronavirus 2019—nCoV contains a furin-like cleavage site absent in CoV of the same clade. Unité des Virus Émergents, Aix Marseille Université, CNRS, AFMB UMR 7257, Marseille, France. Antiviral Research Elsevier. April 2020.
Roman Wölfel et al. (April 2020). Virological assessment of hospitalized patients with COVID-2019. Nature. https://www.nature.com/articles/s41586-020-2196-x.

Clinical Assay of PHT Research Ethics Committee. Andalusian Health Service. Reina Sofia Hospital. Cordoba. Spain. Minute No. 238 ref 2717, with protocol code HDR-2015. PHT clinical trial Influence of HT on lipid profile, carbohydrate metabolism and endothelial function in over 65 years. Ethics Committee Opinion. Gregorio Jurado Cáliz, President of the Ethics Committee, certifies. In Spanish: Comité de Etica de la Investigacion. Servicio Andaluz de Salud. Hospital Reina Sofia. Cordoba. Spain. Acta no 238 ref 2717, con código de protocolo HDR-2015. Ensayo clinico PHT Influencia del HT sobre le prefil lipidico, el metabolismo de los hidratos de carbono y la función endotelial en mayores de 65 años. Dictamen Comité de Etica. Certifica Gregorio Jurado Cáliz, Presidente del Comité de Etica. 2015.
STUDY OF CHRONIC TOXICITY OF MASLINIC ACID. Antonio Zarzuelo & Jesús M. Zúñiga. Dept. Pharmacology. UGR University of Granada, OECD TG 452, BIOMASLINIC SL. (Original in Spanish) ESTUDIO DE LA TOXICIDAD CRÓNICA DEL ÁCIDO MASLINICO EN RATAS. Antonio Zarzuelo & Jesũs M. Zúñiga. Dept. Farmacologia. UGR Universidad de Granada. OCDE TG 452, BIOMASLINIC SL.
Alberto Sarmentero, P.Eng, P. Sc, Raul Montero, P.Eng. et al. (January 2020) GIBIOMED Biomedical Corporate Brochures. Websites: Gibiomed. com, Idroxy.com and Dalphie.com.
CRAG NEWS, (April 2020) Covid-19: How plant biotechnology can help, https://www.cragenomica.es/crag-news/covid-19-how-plant-biotechnology-can-help.

### SUMMARY OF THE INVENTION

Various embodiments of the present invention relate to the field of drugs and bio-affecting compositions which are generally capable of preventing, alleviating, treating, or curing abnormal and/or pathological conditions of the living body, by such means as: (a) protecting the good health conditions of a living body by attracting, disabling, inhibiting, killing, modifying, repelling and/or retarding a virus or micro-organism in the case of preventing an infection (b) destroying a virus or a parasitic organism; (c) limiting the affecting of the disease by altering the physiology of the virus or parasite. In accordance with various embodiments, the present subject matter pertains to pharmaceutical drug compositions targeting various therapeutic objectives and their methods for preventing or treating Covid19 and/or other diseases and certain health conditions including the ones associated with inflammation and/or oxidative stress. They include a Triterpenic compound (hereinafter TMA) with natural Maslinic acid as the main component and other lipophilic phytochemicals obtained from Olive trees and a Polyphenolic hydrophilic compound (hereinafter PHT) with natural Hydroxytyrosol as the main component, all that hereinafter TMA-PHT complex, duly registered as OLIVEVIR™ as well as the methods, elements, combinations, dosage and adjuvant products to improve their solubility, bioavailability and other features for the effective treatment of Covid19 and others diseases through their administration thereof.

This OLIVEVIR™ complex with strong anti-viral activity includes: 20 key phytochemicals extracted with patented natural procedures from the terpene fraction TMA—of these Olive trees, together with a concentrate of 160 polyphenols from their PHT fraction, and this set of natural drugs TMA+PHT® with its synergistic supporting components has been shown to be active in at least 7 key therapeutic targets of Covid19 syndrome that cover preventive, anti-inflammatory and anti-thrombotic and other strategic targets with no toxicity or side-effect problems. This composition is presented for the first time in a highly bioavailable complex for effective absorption by human cells.

Each ingredient of this complex contributes to the activation of specific pathways in humans—and some animals—in unique ways, leading also to up-regulation of cytoprotective genes, as well as effective protection of cells against oxidative stress without side effects. Our team has completed in vitro and in vivo studies with animals in Europe Union, and the drug has already been proven to be safe in its official Toxicology tests that determined its non-toxicity. All OLIVEVIR components have been also authorized by both the FDA and EMA (European Medicines Agency).

### BRIEF DESCRIPTION OF THE DRAWING FIGURES

The present invention will hereinafter be described in conjunction with the appended drawing figures, wherein like numerals denote like elements, and:

FIG. 1 is a schematic view of the contact in the membrane of a human cell of an individual SARS-CoV-2 virus using its “s” (spike) protein with the ACE2 receptor at its Furin cleavage site and TMPRSS2 (transmembrane protease serine 2) for penetrating the human cell. TMA+PHT (OLIVEVIR™) of this invention has a strong serine protease inhibitor effect for the treatment of the Covid19 disease;

FIG. 2 illustrates how TMA+PHT (OLIVEVIR™) of this invention complements antibody activity preventing TMPRSS2 from activating the viral spike protein through protease cleavage;

FIG. 3 is a schematic view of how the mechanism of SARS-CoV-2 virus works by attacking and replicating itself into human cells;

FIG. 4 illustrates the usage of TMA+PHT (OLIVEVIR™) of this invention in the 3 clear phases recognized in the case of a severe COVID-19 infection. At first, patients rapidly develop dyspnea due to fluid in the lungs, as it all starts with a problem of fluids. About nine days after infection, an inflammatory reaction occurs in the lungs, which can further aggravate the situation. Some of the patients who recover after an ICU stay develop thrombosis and scarring of the lungs due to the long-lasting fluid, making recovery difficult;

FIG. 5 depicts the molecular structure of Hydroxytyrosol, the main and most active component of the PHT, the (Hydrophilic) Polyphenolic compound of the Olive used in this invention that includes 160 additional phenols;

FIG. 6 illustrates the molecular structure of Maslinic Acid, the main and most active component of the TMA, the (Lipophilic) Triterpenoid compound of the Olive leafs and fruits (surface), used in this invention; and;

FIG. 7 is a schematic view of the main groups of bio-active organic phytochemical components produced by long-living Olive trees with plenty of new biomedical applications. Extreme hydrophilic phytochemicals (polyphenols) are located in the right side, and extreme lipophilic ones at the left of the figure. Many of them are included in the TMA+PHT complex (OLIVEVIR™) of this invention.

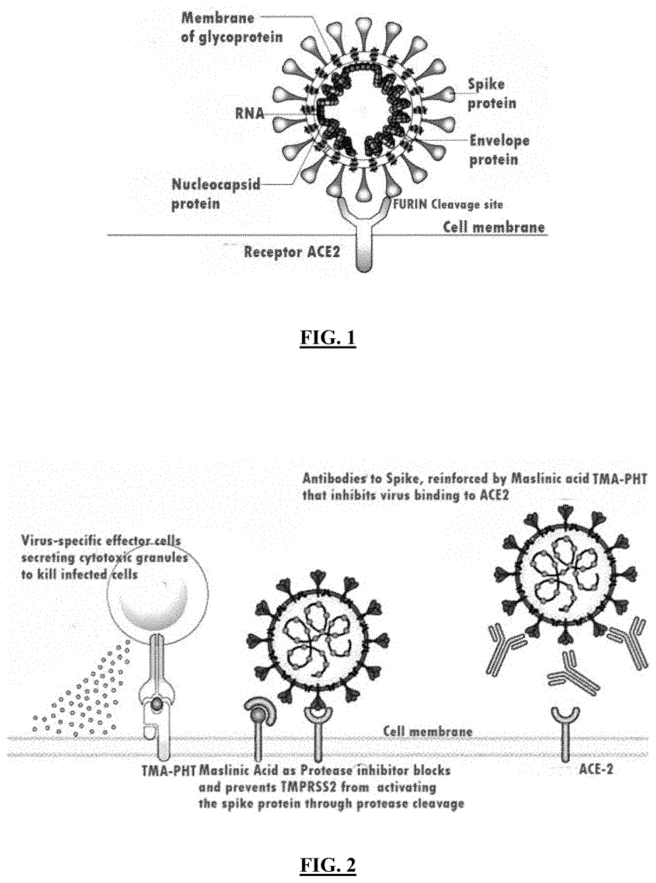

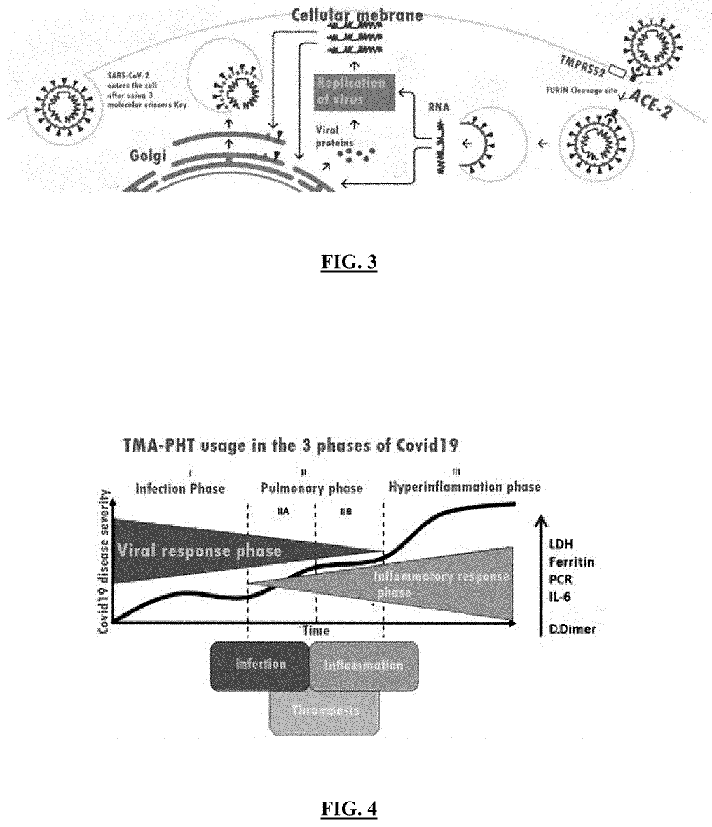

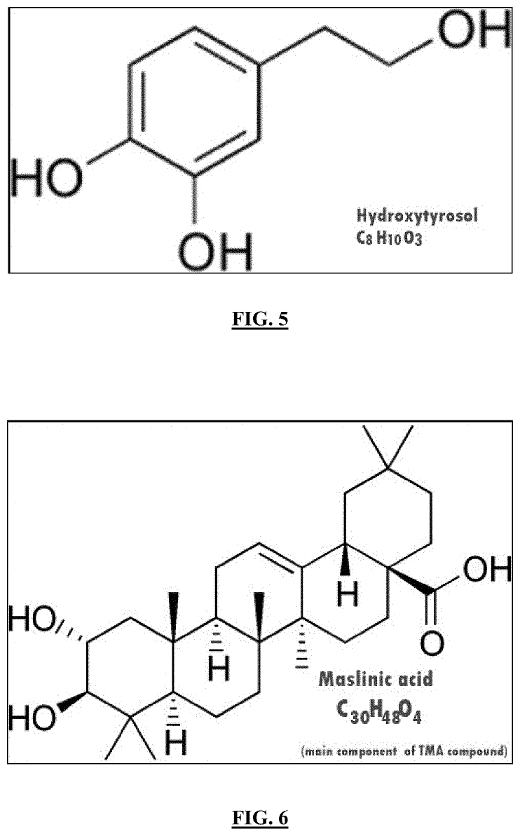

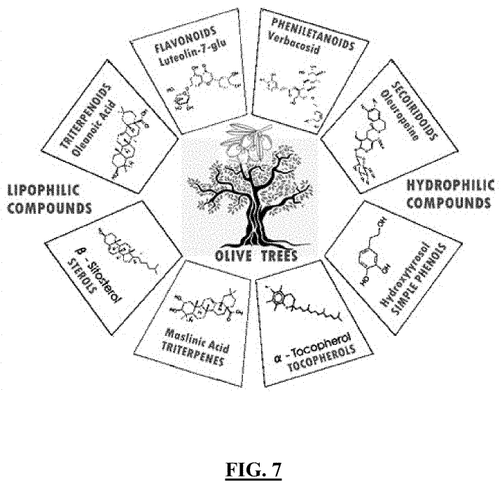

### DETAILED DESCRIPTION OF PREFERRED EXEMPLARY EMBODIMENTS

The present subject matter relates to compositions and usage of drugs containing several bioactive elements for preventing or treating certain health conditions. As a preliminary matter, it will be understood that the following detailed description is merely exemplary in nature and is not intended to limit the inventions or the application and uses of the inventions described herein. Furthermore, there is no intention to be bound by any theory presented in the preceding background or the following detailed description. In the interest of brevity, conventional techniques and components related to viruses and like may not be described in detail herein.

Briefly, by extracting various components of the Triterpenic lipophilic fraction and the different phenols of the Polyphenolic Complex mainly from the fruits, seeds and leaves of long-living olive trees, we recover these phytochemical principles using mechanical (no toxic) extraction technology and proceed to concentrating them separately with high pharmacological standards, and so we obtain our TMA concentrate compound by one side including Maslinic (2-alpha, 3-betadihydroxy-28-carboxyloleanene) as a main component, an acid little distributed in nature, having been detected in a dozen plants but only commercially extracted from Olive trees, Oleanolic acid and some Ursolic acid with minority amounts of the rest of the olive lipophilic fraction described below, and by other side by using another different industrial procedure we recover a highly concentrate PHT compound that later we mix to form TMA-PHT complex: (C30H48O4 Maslinic acid mainly-based Triterpenic compound and Hydroxytyrosol mainly-based C8H10O3 Polyphenolic compound) as drug compositions or pharmaceutical product(s), to be used in several diseases disclosed below, thanks to its Anticancer, Anti-proliferative, Antioxidant, Immunomodulatory, Antimicrobial, Neuroprotective, Lipid lowering and Vaso-modulator effects, while inhibits Cholesterol acyltransferase, Glycogen phosphorylase, Topoisomerase D NA, DNA polymerase and Elastase, preventing from oxidative stress, and—as a viral medicine—it was officially tested in clinical trials for HIV virus with positive results. Since its safety has been already fully tested, the official phase-III Clinical Trial in Europe Union is now starting to precisely measure the range of clinical effectiveness in each of the 7 Covid19 therapeutic targets against which OLIVEVIR molecules are active.

COVID-19 disease primary objectives are the human glandular epithelia of the respiratory and digestive tracts, but the virus has also tropism for the endothelia. This viral infection is associated with a systemic inflammatory and thrombotic immune response, acute interstitial pneumonia and coagulation activation both at the pulmonary level and in the rest of the organs. As described further below TMA-PHT complex, namely OLIVEVIR™, is an active composition in these key therapeutic targets related to COVID19 disease.

On the other hand, each ingredient of the TMA-PHT complex (OLIVEVIR™) contributes to the activation of specific pathways of humans (and some animals) in unique ways, which leads to upregulation of cyto-protective genes and protection of cells against oxidative stress with no side effects reported (as determined in the official toxicity report of this invention), therefore it is recommended the intake of TMA-PHT complex, also as a dietary supplement in prophylactic maintenance doses to promote healthy aging.

TMA+PHT Complex or OLIVEVIR™ Medical Targets

TMA+PHT is a serine protease inhibitor for the treatment of the Covid19 disease caused by this SARS-COV-2 virus, the way it was previously proven positively in the trials performed with other viruses, such as HIV, (since like HIV, the coronavirus uses a serine protease). Inhibition of TMPRSS2 (transmembrane protease serine 2) partially blocked infection by SARS-CoV and Human coronavirus NL63 in HeLa cell cultures and significantly reduces the infection of Calu-3 lung cells by SARS-CoV-2, the virus responsible for COVID-19.

TMA acts also as a non-steroid anti-inflammatory drug, less radical than corticosteroids but without its problems or side effects (risk of cystic fibrosis, etc.) since it acts selectively on Interleukin IL6 (among others), inhibiting it, and this medicine also has an important immuno-modulating effect.

Specific action in relation to COX-2 (Enzyme that accelerates the formation of substances that cause inflammation), Prostaglandins (cellular mediators that play an important role in regulating the allergic inflammatory response), TNF-alpha biomarker (inflammatory route biomarker), etc.

About one third of people who died from COVid19-related causes at global scale passed away because the virus overwhelmed the immune system; while roughly another third portion did perish because the of immune response to secondary bacterial infections, usually in the lungs; and the remaining third was due to the failure of one or more organs.

OLIVEVIR™'s TMA-PHT applications to fight COVID19.

The usage/intake of these drug compounds include the following applications:

1. Preventive elements to avoid Covid-19 infection, also taking advantage of a very low risk of rejection by the patient due to the null toxicity of this TMA-PHT complex, according to the official ad hoc Toxicity study.

2. Direct treatment of Covid-19 viral infection: Every specific place that any SARS-CoV-2 protein interacts with a human protein is a potential drug-able pharmacological focus site, and so the several elements of this multi-drug cocktail TMA-PHT could act on different targets.

3. Effective treatment of inflammation, especially critical during storm episodes of molecular mediators of inflammation such as quinines, especially cytokines and/or bradykinin in humans —triggered by Covid-19.

4. Treatment (and prevention) of Thrombosis. The fact that coagulopathy occurs in patients with COVID-19 has promoted antithrombotic strategies, especially in patients who are admitted to the ICU and show organ damage or ischemic episodes. Although the best antithrombotic strategy has not yet been established, it seems that our PHT compound along with low molecular weight heparins—LMWH—at prophylactic or intermediate doses could be recommended when D-dimer values are higher than normal. Therapeutic anticoagulation is normally reserved for cases in which a clear local or systemic thrombotic pathology is seen.

5. To this list of applications it must be added, even for those already infected with COVID-19—whether or not they are symptomatic—the action of TMA-PHT as an antiplatelet aggregator, often resulting in a preventive measure in these cases, provided the anti-aggregating capacity of TMA-PHT, altering blood coagulation by acting in the first stage of the primary hemostasis of platelet aggregation process and therefore preventing the formation of thrombi or clots inside the arteries.

6. OLIVEVIR also has a positive effect on the intestinal microbiota that in-turn improves the human immune system. It is well documented that human gut microbiota plays a critical role in the immune system and therefore its composition could affect the vulnerability and outcomes of COVID-19 disease. The positive effect of the TMA-PHT® complex on human intestinal microbiota has been established mainly by the University of Jaen. Spain, where TMA-PHT resulted in a change in the intestinal bacterial flora associated with an anti-inflammatory microenvironment, characterized by a decrease in Enterococcus, Staphylococcus, Neisseria and Pseudomonas bacteria, an increase in the Firmicutes/Bacteroidetes ratio and the maintenance of the amount of Akkermansia. This positive anti-inflammatory effect of TMA-PHT on the microbiota, improving the immune system, also resulted in an additional protective factor for other pathologies, so that it also proved to be effective inhibiting the development of colorectal cancer.

7. Post-COVID19: Active elements to fight PPCS (persistent post-COVID-19 syndrome). Among survivors of COVID19, the presence of (PPCS) is a common finding. PPCS has one or more symptoms including fatigue, dyspnea, memory loss, sleep disturbances, and difficulty concentrating. Recent clinical trials also show a biological acceleration of aging and telomere shortening, with a constant increase in biological age in the post-COVID-19 population, which determines a significant Delta-Age acceleration. In addition, the expression of ACE2 decreased in post-COVID-19 patients, thus some epigenetic alterations seem to be associated also with this condition.

OLIVEVIR, and specially its PHT polyphenolic fraction, rich in hydroxytyrosol, has proved to be the strongest antioxidant known today in nature (3-4 times more effective than coenzyme Q10) resulting in proven anti-aging elements with a remarkable therapeutic potential to fight PPCS.

Other common features seen in persistent post-COVID-19 syndrome (PPCS) include increased circulating troponin T and brain natri-uretic peptides (suggesting the presence of myocardial damage with possible activation of a remodeling process). In addition, reduced cardiac contractility and alteration of fibrinogen pathways can increase the risk of blood clotting and pulmonary embolism. Clotting problems have also been observed in post-COVID-19 survivors and in PPCS patients, who are routinely prescribed blood thinners. Despite the variety and importance of symptoms reported by numerous COVID-19 survivors with or without PPCS, ad hoc therapies including OLIVEVIR and valuable biomolecular markers to monitor this condition are still lacking.

As a reference trial, pre-treatment of MDCK (Madin-Darby Canine Kidney cells) with PHT did not affect the propagation of H9N2 virus subsequently inoculated onto the cells, implying that PHT targets the virus but not the host cell. H9N2 virus inactivated with PHT retained unaltered hem-agglutinating activity and bound to MDCK cells in a manner similar to untreated virus. Neuraminidase activity in the PHT-treated virus also remained unchanged. However, in the cells inoculated with PHT-inactivated H9N2 virus, neither viral mRNA nor viral protein was detected. Electron microscopic analysis revealed morphological abnormalities in the PHT-treated H9N2 virus. Most structures found were atypical of influenza virions (complete viral particles constituting the infective form of a virus), and localization of hemagglutinin was not necessarily confined on the virion surface. These observations suggest that the structure of H9N2 virus could be disrupted by PHT. Also, the set of smaller-molecules of the polyphenolic compound PHT, alone or as a part of the TMA+PHT compound, inactivated influenza A viruses in the trials including H1N1, H3N2, H5N1, and H9N2 subtypes. PHT also inactivated Newcastle disease virus but not bovine rotavirus, and fowl adenovirus, suggesting that the mechanism of the antiviral effect of PHT might require the presence of a viral envelope.

Cytokines are small proteins secreted by immune cells that have an effect on other cells. Examples are interferons (IFNs), TNF alpha, interleukins (IL1,6), and transforming growth factors (TGFs). During the course of COVID-19, some patients bring down viral numbers only to rapidly descend into a state of shock involving hyper-activation of the immune system and hyper-coagulation in small blood vessels. This rapid and uncontrolled inflammatory signaling cascade, known as a “cytokine storm,” exacerbates the heavy breathing and low oxygenation, and triggers inflammation in major tissues such as the lungs, kidneys, heart, liver and brain. The resulting vascular inflammation is emerging as a main cause of complement-associated microvascular injury and thrombosis in severe COVID-19 cases.

The effects of PHT on the inflammatory mediators —mainly consisting of peptides whose biological function is directed to attract immune cells to damaged tissues with participation in the production and modulation of pain sensation: Bradykinins, Cytokines and/or Chemokines—were evaluated in several studies in which the PHT, the smaller molecule pool of the compound TMA-PHT—has been shown to inactivate influenza A viruses in assays, including the H1N1, H3N2, H5N1 and H9N2 subtypes. PHT also inactivated Newcastle disease virus, but not bovine rotavirus and avian adenovirus, suggesting that the mechanism of the antiviral effect of PHT might require the presence of a viral envelope. PHT inhibited the production of nitric oxide NO and prostaglandin PGE₂ with strong anti-inflammatory activity, with a sharp decrease in the secretion of cytokines (IL-1 α, IL-1 β, IL-6, IL-12, TNF-α) and chemokines (CXCL10/IP-10, CCL2/MCP-1). and dependently reduced the concentration of nitric oxide genes. HT was identified as the main bioactive compound. The data provides a molecular basis for elucidating the effects of PHT on inflammatory processes. The effects of HT on NO and the production of chemokines point to its impact not only in COVID19 cases but also on other infections or pathologies like chronic inflammatory processes in the endothelium or arthritis.

As recited above, TMA+PHT or OLIVEVIR™ belongs to a class of medications known as protease inhibitors, the same type of drug that is used to treat other viral infections such as HIV or hepatitis C. It works by inhibiting an enzyme from the COVID-19 virus that is necessary for the virus to replicate in human cells. This medicine is aimed at those who might have been infected and could prevent the disease from progressing and no longer require these patients to have to be admitted to a hospital. The binding of the spike protein that enables SARS-CoV-2 to enter target cells is dependent on the host cell receptors angiotensin-converting enzyme (ACE2), and CD147. It also requires priming by a transmembrane enzyme encoded by the gene TMPRSS2, which involves cleavage of the spike protein to allow fusion to the host cell membrane. OLIVEVIR's TMA+PHT complex down-regulates the expression of ACE2, and TMPRSS2, and up-regulates a potent inhibitor of TMPRSS2 called PAI-1 (plasminogen activator inhibitor-1). TMA+PHT Complex treatment might diminish the ability of SARS-CoV-2 to bind to a host cell, as a result of less ACE2 and TMPRSS2 on the cell surface, and as a result of the increase in plasma PAI-1, which would inhibit the remaining TMPRSS2.

TMA's anti-proliferative activity likely comes from the induction of an oxidative apoptotic pathway, causing cell cycle and cytoskeleton alterations. Also TMA's Maslinic acid has been found to get a strong reduction of pro-inflammatory cytokine generation in murine macrophages, while it has been proven in the trials to inhibit the spread of the HIV virus by inhibiting the replication of a primary HIV-1 isolate as well as decreased the cytopathic effect and p24 antigen levels in MT2 cells and attenuate intracellular oxidative stress via inhibition of NO and H2O2 production.

Furin scissors and TMPRSS2 (transmembrane protease serine 2)—an enzyme that in humans is encoded by the TMPRSS2 gene—are key in the COVID 19 infection. This gene encodes a protein that belongs to the serine protease family. The encoded protein contains a type II transmembrane domain, a receptor class A domain, a scavenger receptor cysteine-rich domain and a protease domain. Serine proteases are known to be involved in many physiological and pathological processes. This TMPRSS2 human gene was demonstrated to be up-regulated by androgenic hormones in some cells and down-regulated in androgen-independent tissue. The protease domain of this protein is thought to be cleaved and secreted into cell media after auto-cleavage. Alternatively, spliced transcript variants encoding different isoforms have been found for this gene.

Some coronaviruses, e.g. both the SARS coronavirus of 2003 and this SARS-CoV-2 are activated by TMPRSS2 and can thus be inhibited by TMPRSS2 inhibitors. SARS-CoV-2 uses the SARS-CoV receptor ACE2 for entry and the serine protease TMPRSS2 for S protein priming. A TMPRSS2 inhibitor like TMA-PHT blocks the entry and constitute a treatment option alone or combined with other inhibitors. However, the spike of SARS-CoV-2 is like a 3-part key. The first one binds to the lock: the ACE2 receptor on the human cell, but this binding is not enough to achieve invasion. Viruses need to cut their protein from the spike to activate it and start the attack. The second part of the key deals with the fusion of the virus membrane and the cell membrane. The great novelty of SARS-CoV-2 compared to other former coronaviruses is the occurrence of 12 extra letters inserted into its genome making the third part of the key, the Furin key. This very short genetic sequence seems to be the main culprit for its wide contagiousness and virulence.

Plasma membrane-anchored serine protease facilitates SARS-CoV-2 infections via two independent mechanisms, proteolytic cleavage of ACE2 (Angiotensin-converting enzyme) receptor which promotes viral uptake, and cleavage of coronavirus spike glycoproteins which activates the glycoprotein for host cell entry. It cleaves and activates the spike glycoproteins of human coronavirus 229E (HCoV-229E) and human coronavirus EMC (HCoV-EMC) and the fusion glycoproteins FO of Sendai virus (SeV), human meta-pneumovirus (HMPV), human parainfluenza 1, 2, 3, 4a and 4b viruses (HPIV) and is essential for the spread and pathogenesis of influenza A virus (strains H1N1, H3N2 and H7N9); involved in proteolytic cleavage and activation of hemagglutinin (HA) protein which is also essential for viral infectivity. The other human receptor is CD147, which has an affinity for the same virus surface protein S as ACE2, although it is not yet clear if both receptors are equally important or if CD147 is secondary.

Serine protease enzymes have a variety of functions in the body, and so serine protease inhibitors like TMA have a diverse range of uses (by Blue Singularity S.L. Spain, a subsidiary of GIBIOMED.com). This drug has been positively tested also in the treatment of some forms of cancer and is also effective against some viral infections including HIV, and we are studying it for potential use for inhibiting fibrosis in liver or kidney disease and pancreatitis.

Antiviral, Anti-Inflammatory and Immunological Activity of TMA-PHT Complex

According to a clinical trial carried out on the pharmacokinetics of TMA, and its components MA (maslinic acid: 2-alfa,3-betadihydroxy-28-carboxiolean-12-eno) and OA after ingestion of olive oils (OO) with high and low Triterpene acids content, and specifically the effect of these triterpenes on endothelial function. A double-blind, dose-response, randomized, crossover nutritional intervention was performed at Univ. of Granada in healthy adults, and resulted that MA and OA values became increased in the biological fluids in a dose-dependent manner. The bioavailability of MA was higher than that of OA, and the consumption of these TMA penta-cyclic triterpenes was associated with improved endothelial function.

Similar Triterpenes of this kind obtained from the roots of some Asian plants have a history of medicinal use in many Asian countries. In our case TMA's Pentacyclic triterpenes have proven to possess strong antioxidant properties, as they prevent lipid peroxidation and suppress generation of superoxide anions. TMA's Maslinic acid is useful in modulating the immune response; Further measuring research will be required to rate the immunomodulatory behavior of this triterpenoid and to characterize the mechanisms underlying the biphasic nature of some aspects of the inflammatory response. Our research groups at our Biotech company Blue Singularity S.L. Spain (formerly Biomaslinic Inc.) and the Labs of our faculty of Sciences of Granada University are methodologically carrying out further molecular biology tests in course.

During the Covid19 Pandemic situation obviously, besides the existing vaccines, there has been an urgent need for curative drugs, most of them based on antiviral compounds that can treat the infection. Other drugs that are being administered to patients are intended to control or modulate the exaggerated response of the immune defense against the virus itself, including interferons, corticosteroids, monoclonal antibodies for the interleukin receptor IL-6, inhibitors of complement activation etc.

Current management of COVID-19 is still mainly supportive, and respiratory failure from acute respiratory distress syndrome (ARDS) is the leading cause of mortality. Secondary haemophagocytic lympho-histiocytosis (sHLH) is an under-recognized, hyper-inflammatory syndrome characterized by a fulminant and fatal hyper-cytokinaemia with multi-organ failure. In adults, sHLH is most commonly triggered by viral infections and occurs in 3.7-4.3% of sepsis cases. Cardinal features of sHLH include unremitting fever, cytopenias, and hyper-ferritin-anemia; pulmonary involvement (including ARDS) occurs in approximately 50% of patients. A cytokine profile resembling sHLH is associated with COVID-19 disease severity, characterized by increased interleukin (IL)-2, IL-7, granulocyte-colony stimulating factor, interferon-γ inducible protein 10, monocyte chemoattractant protein 1, macrophage inflammatory protein 1-α, and tumor necrosis factor-α.

Effects of TMA-PHT (OLIVEVIR). In Course Research:

Regarding the effect of TMA on serine proteases: Previously, our group BLUESINGULARITY SL. has worked on the effects that Maslinic acid and some chemical derivatives have on viral serine protease activity (2014. European Journal of Medicinal Chemistry, 74: 278-301). In accordance with what has been done, we are deepening this path using mimetic models of the SARS-CoV-2.

Anti-inflammatory effect of TMA. Effect of maslinic acid on the levels of cytokines, interleukins, cell growth factors, type TNA-α, NAFT, NFKB, etc., in specific cell lines for these studies: how it affects the levels and activity of related enzymes like COX-2.

TMA-induced intracellular signaling pathways. Deciphering the different intracellular signaling pathways caused by TMA in its anti-inflammatory activity, such as intracellular signaling routes: JAK-STAT, MAPK, etc.

Effect of TMA on membrane receptors. SARS-CoV binds to angiotensin-converting enzyme 2 (ACE2) while MERS-CoV binds to dipeptidyl peptidase 4 (DPP4). (ACE2 (e.g., pneumocytes, enterocytes) or DPP4 (e.g., liver or lung cells including Huh-7, MRC-5, and Calu-3). Measuring the levels and functionality of the different membrane receptors that uses the virus for its penetration, using for it appropriate cell lines.

Effect of TMA-PHT Complex on cellular immunogenic capacity in the appropriate cell lines: Effect of maslinic acid on the production of immunoglobulins, with special emphasis on M (IgM) immunoglobulins, characteristic of the primary response, and Immunoglobulins G (IgG) characteristic of the secondary and memory-responsible immunological responses.

Synthesis of new chemical derivatives of TMA, such as occurred with oleanolic acid, with greater anti-inflammatory and immunological finality.

Description of the Components and its Activity

TMA and PHT compounds of OLIVEVIR have been generated by wild Olive plants (acebuche) along millions of years throughout their evolutionary mechanism of self-protection against attacks by many kinds of viruses and other pathogens. Of all these activities, what interests us most is its potent anti-inflammatory capacity, since as said, it inhibits the formation of COX2 and some pro-inflammatory interleukins. So, we are pioneering its usage in human health applications. We have also discovered that OLIVEVIR's TMA-PHT has an important cellular regenerative capacity at low doses and at high doses it is a potent selective inducer of apoptosis in cancer cells. Its most notable activity in recent scientific studies is the correction of GEN P53, as a guardian of the genome. P53 is the gene that acts to eliminate mutated cells, so that when P53 is altered, the body has no endogenous defense to fight against carcinogenic processes. P53 is a tumor suppressor protein. In the human species, the p53 or TP53 gene, also called the “genome gatekeeper”, is found on the short arm of chromosome 17 and encodes a nuclear transcription factor of 43.7 KDa. This p53 protein plays a crucial role in the regulation of cell proliferation and in the cell's response to stress stimuli; p53 induces both the stop of the cell cycle that prevents the replication of damaged DNA, and the activation of apoptosis to eliminate “defective” cells.

TMA, the Triterpenic phytochemical lipophilic compound extracted from various parts of the olive tree (mainly fruits, leaves and seeds) whose biochemical components are subsequently concentrated, consisting in addition to Maslinic acid (the most abundant triterpene in olive trees, averaging 40% of all Triterpenes and Triterpenoids fraction), Oleanolic acid (ranging >15%), and small concentrations or traces of other natural components such as Erythrodiol, Beta-amirin, Beta-amirone, as well as triterpenes derived from Lupane (Lupeol, Lupone), derivatives of Ursan (Ursolic acid, Uvaol) and derivatives of Taraxane (Taraxerol) completing the remaining fraction.

As recited above, the TMA (alone or included in the TMA-PHT complex) could be used as serine proteases inhibitor for the treatment of COVID19 and other diseases caused by viruses, the way it was used successfully against HIV (acquired immunodeficiency virus). To reduce the harmful effects of exposure to pathogens and DNA-damaging agents, including viruses, the olive trees have evolved a complex network of genome stability pathways including the generation of these acids as well as the PHT components of olive polyphenols with different protective properties.

The effectiveness and success of the biochemical warfare that Mediterranean olive trees have achieved in their survival fight against viruses and other pathogens allows such olive trees to achieve a healthy lifespan of more than 3,000 years potential, while some of such olive trees-extracted biochemical components result in suitable preparations for the treatment of certain diseases caused in humans and animals by other viruses or pathogens.

PHT, the polyphenolic hydrophilic phytochemical compound, also extracted from olive trees includes 160 polyphenols (with a separate and different extraction and concentration technology), which includes Hydroxytyrosol (the most abundant polyphenol in olive trees, averaging 40%), Oleuropein, Tyrosol and smaller amounts of minor olive polyphenols from the Olive Phenolic fraction including Oleocantal, Oleacein, Hydroxypheniletanol, Quercetine, Luteoline, Rutine, Verbacosid, Floridzine, as well as Galic, Clorogenic, Vanilic, Cafeic, Cumaric, Elenoic and Transcinamic acids, and very small or even trace amounts of other olive polyphenols totaling 160 known and not very well known phenols.

Hydroxytyrosol (HT) comes from the hydrolysis of Oleuropein, a bitter glycoside of the olive fruit. Oleuropein is hydrolyzed giving rise to its components: oleuropein aglycone, HT and Elenoic acid. Among those the most relevant are oleuropein, oleocantal, oleacein, and tyrosol. The fundamental activity of these compounds is to protect the fruit from oxidation against oxidative stress, including the activity of UV radiation from the sun that causes cell mutations and accelerated skin aging. Of all these components the best known is hydroxytyrosol, that is also produced by humans and few other mammals in very small scale and prevents oxidation of blood fats and therefore prevents oxidation of cholesterol and with it, the formation of thrombi, in turn protecting the endothelial system. It contributes to repairing the changes in the DNA of the skin cells produced by UV radiation, prevents the passage of hepatic steatosis to steo-hepatitis, has an important neuronal activity so it has preventive effectiveness in Alzheimer and Parkinson, regulates the level of blood glucose and has proven activity against diabetic retinopathy. The activity of this product is so important that our body has learned to make it from endogenous dopamine and has been found in the iris of the human eye in people who had not previously consumed it. The presence of potent anti-cancer azelaic acid in the skin cannot be forgotten.

The mixture of both groups of compounds TMA and PHT: TMA-PHT Complex of OLIVEVIR, with synergistic properties, or any of its components or combinations, as drugs and/or pharmaceutical products for the preparation, alone or in combination with other agents, of medicines for the treatment of human or animal diseases caused by any agent, including agents that cause COVID19, is characterized in that the product has several medical effects including the effect as an inhibitor of protease.

The antimicrobial properties of TMA's oleanolic and maslinic acid have been tested in vitro: After synthesizing and studying fourteen oleanolic and maslinic acid derivatives, the research team found two, called OA-HDA and MA-HDA, that exceeded the antimicrobial activity of the original compounds both in vitro and in an animal model. And most importantly: the bacteria did not demonstrate the ability to generate resistance to either compound. This is relevant now when the inappropriate use of antibiotics has resulted in the emergence of bacteria resistant to multiple drugs, that are capable of surviving despite the use of such drugs.

On the other hand, Cytokines play a key role in controlling the immune response and cell growth. Many cytokines work by binding and activating type I and type II cytokine receptors. These receptors, in turn, depend on Janus kinase (JAK), which are enzymes for signal transduction. Therefore, drugs that inhibit JAK activity block cytokine signaling. Such TMA-induced intracellular signaling pathways Janus kinase inhibition (JAK) could affect both inflammation and cellular viral entry in COVID-19. As with previous viral pandemics (SARS and MERS), corticosteroids are not routinely recommended and may exacerbate COVID-19-associated lung injury. However, in hyper-inflammation, immunosuppression is likely to be beneficial. The IL-1 blockade test (anakinra) in sepsis showed a significant survival benefit in patients with hyper-inflammation, without increased adverse events. Predictors of fatality from a recent retrospective, multicenter study of 150 confirmed COVID-19 cases included elevated ferritin (mean 1297.6 ng/ml in non-survivors vs 614.0 ng/ml in survivors; p<0.001) and IL-6 (p<0.0001), indicating that such high mortality could be partially due to virally driven hyper-inflammation.

TMA acts selectively on Interleukin IL6, inhibiting it, thus interfering with the JAK-STAT signaling pathway. This inhibitor has therapeutic application in the treatment of inflammatory diseases with an important immuno-modulating effect and so, it could be used alone or combined with other JAK inhibitors that already reached clinical trials like Tofacitinib, a specific inhibitor of JAK3 (IC50=2 nM) thus blocking the activity of IL-2, IL-4, IL-15 and IL-21. Tofacitinib to a lesser extent also inhibits JAK1 (IC50=100 nM) and JAK2 (IC50=20 nM) which in turn blocks IFN-gamma and IL-6 signaling and therefore Th1.1 cell differentiation.

Hyper-inflammation screening tests using laboratory trends (e.g. increased ferritin, decreased platelet count, or globular sedimentation rate) and HScore11, are used to identify the subgroup of patients for whom different levels of immunosuppression could improve their health and diminish mortality rate. Therapeutic options include steroids, intravenous immunoglobulin, selective cytokine blocking and JAK inhibition.

Blood clots are also an important player in the severity and mortality of COVID-19 disease. This virus affects vascular biology and that is why we see that the risk factors are vascular: diabetes, obesity, age, hypertension: The virus can directly attack the lining of the heart and blood vessels, also rich in ACE2 receptors. This tendency to blood clotting transforms some mild cases into life-threatening emergencies: this virus acts like no other pathogen that has been described.

Among 184 COVID-19 patients in a Dutch ICU, 38% had blood that clotted abnormally and nearly a third already had clots, according to an Apr. 10, 2020 article from Thrombosis Research. Blood clots can separate and end up in the lungs, blocking vital arteries and causing a pulmonary embolism. Blood clots in the arteries can also lodge in the brain and cause a stroke. Many patients have very high levels of D-dimer, a by-product of blood clots.

Nosocomial hospital infections: About 5% of all patients acquire an infection during their hospital stay. They are generally caused by the Staphylococcus aureus bacteria and are associated with increased morbidity and mortality, as well as a great economic impact on the health system. Most nosocomial infections originate during invasive procedures, such as the application of catheters or prostheses. Unfortunately, the surfaces of these devices favor the formation of biofilms, bacterial ecosystems with rates of resistance to antibiotics up to 1000 times higher than normal bacteria.

The only effective treatment against a biofilm infection is medical device removal and long-term antibiotic therapy, which can promote antibiotic-resistant bacteria. In such a situation, the compounds of TMA-PHT would be very useful, since their antimicrobial activity in the biofilms of the catheter has also been tested, with positive results. To create therapeutic compounds, we use TMA whose derivatives require very little chemical transformation, so getting them from the original natural olive tree sources is relatively simple, allowing designing compounds of new antimicrobial drugs that act effectively without the danger of causing resistance to antibiotics.

Besides the above applications, the urgent object of the invention is the utilization of OLIVEVIR's TMA-PHT complex, or any of its compounds, either alone or in combination with complementary or synergistic drugs, to get—or be a part of—an effective treatment of several conditions and diseases, but specially the COVID19 disease caused by SARS-CoV-2 virus that has been a challenge for our global scientist community.

SARS-CoV-2 is quite unusual. Respiratory viruses commonly infect either the throat or the lungs. Those that are concentrated in the throat cause milder symptoms, but are very easily spread. Viruses that enter the lungs cause more serious illnesses but are much less infectious. SARS-CoV-2 is both highly contagious and potentially very virulent.

This virus, a tiny little ball of about 50-200 nanometers in diameter, it is as small with respect to a human being as a dog with respect to the entire planet Earth. That RNA virus has a very small genome written with combinations of the same four letters. Each of them is the initial of a chemical compound. With these four letters (a, u, g, c) is written the text that has killed so many people since its existence was detected.

After entering a person's body through the eyes, nose, or mouth, SARS-CoV-2 begins to use human cells in the nose and throat to reproduce. It contacts epithelial cells and type 2 pneumocytes and blocks receptors ACE2 and CD147, leaving them open to put its viral RNA inside the cell. The virus hijacks the machinery of the cell, multiplying copies of itself and invading new cells. so as many as 10,000 copies of such viral genetic material can come out of one single cell. In various cases of Covid19, the virus maintains a complex interaction with its host and causes immune hyper-reactivity with unfavorable side effects.

The genetic material—that some RNA viruses inherit from others—works as an instruction book to build the proteins that make up SARS-CoV-2 genome is only 8 Kb or about 30,000 letters long, but however, with enough instructions to penetrate a cell, hijack its machinery, and make thousands of copies of itself. The entire SARS-CoV-2 genome would fit on four newspaper pages. About 4,000 letters of that text contain the guidelines for the human cell to manufacture the virus's main weapon: its spike protein, the key with which new viruses will open more and more cells.

Meanwhile, our innate immune system kicks in and various types of cells come into action to defend the organism: macrophages, leukocytes, interferon, NK lymphocytes (called killer cells), etc. The most important are macrophages, which target the viral RNA to kill it, and lead the action of cytokines being released to fighting the invading genome and the blocking action of interferon, so our body can beat the virus. However there is a minority of patients (less than 10%) with wicker immune systems whose response by cytokines is magnified to trigger the above mentioned cytokine storm, an autoimmune reaction posing a threat to the organism itself which causes inflammation in various organs, specially the lungs where this cytokine storm causes an edema, a loss of fluid, which makes breathing capacity much more difficult with respiratory failure, while the interior of the blood vessels become inflamed and thrombi occurs, all that being the main cause of death for COVID-19 patients.

If the human immune system does not counteract SARS-CoV-2 during the initial phase, the virus travels down the trachea to attack the lungs, where it can become fatal. The thinner and more distant branches of the respiratory tree of the lungs are also rich in ACE2 receptors, that are the human cell lock that already used the first SARS Coronavirus much more inefficiently in 2002. The key to the SARS-CoV-2 binds to this lock with an affinity up to 20 times greater than the first SARS. In these cases, the standard procedure treatment is aimed at using respirators, corticosteroids, and heparin to avoid thrombi. But instead of corticosteroids we may use TMA-PHT alone or together with ad hoc controlled monoclonal antibodies to block this cytokine discharge.

The former virus SARS of 2002 took advantage of two scissors present only in some cells, the TMPRSS2 enzymes and cathepsins. But the new SARS-CoV-2 also uses a third scissors which is Furin. The virus releases its genome into the cell and begins to make copies of itself. It does this by tricking the cell, which creates the viral proteins. Its parts are assembled and once multiplied it is ready to infect other cells. The 12 extra letters of the virus create a cut point in the protein of the spicule by this another scissors: Furin, an enzyme present in almost all human cells, which explains its high transmissibility and virulence. Furin makes a first cut of the spike of the new viruses, which already leave the human cell pre-activated for a new invasion. This first cut allows the spike to initiate fusion of the infected cell with another healthy cell, allowing the virus to pass from one to the other without exposing itself to antibodies from outside and continue the process of infection and multiplication.

This seems a decisive factor to explain the SARS-CoV-2 enormous number of casualties worldwide in the last years. Furin—an enzyme that in humans is encoded by the Furin gene—allows the virus to invade cells of the human digestive system and kidneys, not only those of the respiratory system. Ebola itself, the dengue virus and the HIV virus also use Furin in their assaults on human cells.

Some proteins are inactive when they are first synthesized and must have sections removed in order to become active. Furin cleaves these sections and activates the proteins. This insertion (Furin cleavage site) allows the virus to enter a greater variety of cells. This favors the spread of the virus, pre-activated for invasion in infected patients and, therefore, is probably a key for the development of the disease. That furin cut point is one of the reasons that SARS-CoV-2 is so transmissible. The acquisition [by natural mutations] of a furin cut point as in the avian influenza viruses is precisely what makes them highly pathogenic viruses.

Viral buildup triggers a strong response from human's immune system, which sends large numbers of white blood cells, antibodies, and inflammatory molecules to eliminate the threat. T cells attack and destroy the tissue that houses the virus, especially in the respiratory tract and lungs where the virus tends to take root. In most healthy adults, this process works and they recover in a short time. But sometimes the immune system's reaction is too strong, destroying so much tissue in the lungs that they can no longer supply enough oxygen to the blood, resulting in hypoxia and death.

Although the lungs are known as ground zero, their reach can extend to many organs, including the heart and blood vessels, the kidneys, the intestine, and the brain. Covid19 can attack almost anything in the body with devastating consequences.

In other cases, it is not the virus itself that triggers a life-threatening immune response, but a secondary infection that takes advantage of a weakened immune system: Streptococcus, or staph bacteria, usually infect the lungs. This infection could be prevented when possible with TMA-PTA (antiseptic and immuno-modulator) and/or immediately treated once detected with due specific medication. Otherwise, a bacterial infection in the respiratory tract can potentially spread to other parts of the body and blood, including causing septic shock—an aggressive, life-threatening inflammatory response that damages multiple organs.

Normally, oxygen crosses the alveoli into the capillaries next to the air sacs, but as the immune system fights against the invader, the battle itself disrupts the transfer of oxygen. Front-line white blood cells release inflammatory molecules that in turn summon more immune cells that attack and kill virus-infected cells, leaving a residue of fluid, dead cells, and pus. As previously said, it is a reaction called a “cytokine storm” in which the levels of cytokines rise and immune cells begin to attack healthy tissues. Blood vessels leak, blood pressure drops, clots form, and catastrophic organ failure can occur.

In addition to pneumonia, secondary complications of COvid19 range from relatively minor ones, such as ear infections, to much more serious complications, such as inflammation of the heart (myocarditis), the brain (encephalitis), or muscles. (myositis and rhabdomyolysis). They can also include other ailments in which the immune system attacks the peripheral nervous system.

The high morbidity and mortality of this disease seems to be due to this disproportionate inflammatory response to the virus. Several drugs targeting cytokines, including TMA-PHT complex are or will be soon in clinical trials in patients with COVID-19. But it is very important that these drugs do not suppress the immune response that the body needs to fight the virus. The more virulent strains trigger a stronger inflammatory response with much primary viral pneumonia.

The immune system of the elderly is often weakened by a combination of age and underlying diseases that makes them less able to tolerate and recover from the self-attack of the immune system. Most people at increased risk of developing potentially fatal complications are older people and continually exposed health workers.

### STATE OF THE ART

Viruses are mutating all the time. A single virus that infected an animal can multiply exponentially up to a trillion infectious viral particles in its body. We have to expect many new variants of Sars-CoV-2 after DELTA variant. We should never underestimate the ability of a virus to adapt. It is like the celebrated paradox about trillions of monkeys with typewriters that can randomly result one to write a great known poem.

As recited above, Coronaviruses (CoV) are enveloped positive-stranded RNA viruses belonging to the Nidoviralis order, detected in mammals and birds, that are specifically denoted as causing etiologies of upper respiratory tract infections in humans. Two potentially dangerous zoonotic coronaviruses have emerged in the past two decades. The SARS-CoV was responsible for the first outbreak that spanned from 2002 to 2003. The second outbreak occurred in 2012 in the Middle East and was affected by the respiratory syndrome coronavirus Middle East (MERS-CoV).

According to its genome sequence, SARS-CoV-2 belongs to such beta-coronavirus lineage b which also includes SARS-CoV and Bat CoV ZXC21, the latter and CoV ZC45 being the closest to SARS-CoV-2 sharing ˜76% amino acid sequence identity in the Spike protein sequence with SARS-CoV and 80% with CoV ZXC21. They focus also on a specific furin-like protease recognition pattern present in the vicinity of the protein S maturation sites that has significant functional implications for virus entry.

Since early studies it was shown that amounts of pro-inflammatory cytokines in serum (such as IL1B, IL6, IL12, IFNγ, IP10, and MCP1) are increased which were associated with lung inflammation and extensive lung damage in patients with SARS. MERS-CoV infection was also reported to induce higher concentrations of pro-inflammatory cytokines (IFNγ, TNFα, IL15 and IL17). Similarly, Covid19-infected patients also had high amounts of IL1B, IFNγ, IP10, and MCP1, which likely led to activated T-helper-1 (Th1) cell responses. Furthermore, patients who required ICU admission had higher concentrations of GCSF, IP10, MCP1, MIP1A, and TNFα than those who did not require ICU admission, showing that cytokine storm was associated with disease severity. However, Covid19 infection also initiated increased secretion of T-helper-2 (Th2) cytokines (e.g., IL4 and IL10) that suppress inflammation, which differs from SARS-CoV infection.

The SARS-CoV-2 genomic sequence indicates that this zoonotic virus is grouped with lineage b beta-coronaviruses. The SARS-CoV-2 protein S sequence has a specific Furin-like cleavage site absent in the b CoV lineage, including the SARS-CoV sequences. The furin-like cleavage site in the SARS-CoV-2 protein S has implications for viral life cycle and pathogenicity. Campaigns to develop anti-SARS-CoV-2 therapies should include the evaluation of Furin inhibitors.

Furin has been known only since 1990, but it does not seem to be the Achilles heel of the SARS-CoV-2 to attack it with drugs because it has a fundamental role in the human body. Its snips activate the precursors of many of the proteins that carry out the basic tasks for human life. It is an essential enzyme for the virus, but also for the person. Inhibiting Furin would generate toxic effects on cells. It could become a target for drugs against covid-19, but it does not seem the most ideal.

Despite great similarity to the SARS CoV and SARS-like CoV genome sequence, we identified a peculiar furin-like cleavage site in the SARS-CoV-2 Spike protein that lacks the other SARS-like CoVs. The functional consequences of this cleavage site on the viral cycle, the pathogenicity and its possible involvement in the development of antivirals are very relevant.

Efficient person-to-person transmission is a requirement for the large-scale spread of a new virus. The proportion of patients with mild symptoms of disease is another important factor that makes it difficult to identify infected people and prevent the spread of the virus. The identification of transmission chains and the subsequent follow-up of contacts are even more complicated when several infected people remain asymptomatic or slightly symptomatic. A key factor for efficient person-to-person transmission is the ability of the virus to bind to human cells.

The olive tree (Olea europaea) is an evergreen tree native to the Mediterranean that originated about 20-40 million years ago in the Oligocene times at the Mediterranean basin. Along with grain and grapes, they became part of the triad of staple crops that fueled the emergence of more complex human societies.

Since the beginning of the domestication and cultivation of the formerly widespread ancient Mediterranean wild olive trees (so-called acebuche) dates back to the early Iberian, Phoenician, and Greek cultures. Thus, the first known documents written on the olive tree are Mycenaean tablets in mud, from the reign of King Minos and dated around 2500 BC. There are olive trees older than 3000 years still producing olives. Spain holds today around the 50% of Olives and Olive oil industrial production in the World and is leading the research on this peculiar plant. It also holds by far most of the living millenary olives (more than 1000 y.o.) worldwide.

The oil trade obtained in Roman times, extracted from the olive trees of South Spain area (where the largest humanized forest in the world is located) was spread throughout all the western world, and even exported to China. That is to say, this area was already the world's chief olive center since more than 2,000 years ago. So, it represents the largest spot concentration of E Vitamin on the planet (along with Vitamins A, D and K, also very abundant in the Olives). Some of these ancient olive trees are still alive and producing today.

During last years, The University of Granada (Faculty of Sciences), Spain, has patented the industrial use from the by-products of the olive milling in any of its variants (Industrial use of oleanolic and maslinic acids contained in the by-products of olive grinding (P9601652). It has also been patented by the University of Granada the preparation of certain types of drugs against parasitic infections (Use of Maslinic Acid as a Serine Protease Inhibitor for the Treatment of Diseases caused by parasites of the genus Cryptosporidium (P9701029).

Today, Japan and Spain are the countries with the highest lifespan and life expectancy rate in the World, according to the Organization for Economic Cooperation and Development (OECD), and Health at a Glance, the most recent health report of that entity. Besides genetics, healthcare and lifestyle, some of the most important reasons for that extended longevity seem to lay in metabolic advantages resulting in a proper cell preservation, and in the case of Spain the statistical reason seems to lay in the so-called Green Gold: The consumption of certain natural polyphenols and other phytochemicals —most of them exclusive from Olive trees-all present in very small amounts in olive (fruits) and olive oil widely consumed by local population as a key part of their Mediterranean diet.

Plants and pathogens (including viruses) had a long and ancestral fight along all geological times, resulting in victories and defeats for the plants in such a tough “biochemical wars”, while evolutionarily improving their defenses with brand new substances such as the Triterpenic fraction and the Polyphenolic fraction ones that made the Olive trees to win such a battles by managing to destroy their pathogens and get to live a very long life. Some animals —including early humans—eventually took advantage of these plant's elements for their survival by eating and assimilating such phytochemical substances. That's in the origin of medicine.

When a virus like SARS-CoV-2 tries to infect a human, once it manages to overcome the physical barriers imposed by the skin, it faces our innate immune system, a defense system that all animals have. It is made up of four biological weapons: 1) phagocytes, which are white cells (e.g. macrophages) that patrol the tissues of the body cleaning it of garbage, cellular debris and invaders. 2) The complementary system, which is made up of approximately twenty proteins produced in the liver and found in high concentrations in the blood and tissues, these work together to destroy the invaders (make perforations in the protein envelope or membrane of the invaders) and to give the alarm signal to other members of the immune system team. This system is so very much old that even sea urchins that evolved approximately 700 million years ago have it. 3) The alert system of interferons, which are proteins produced by cells that bind to small receptors (keys) of the cell membrane and serve to alert the cell that it will soon be attacked by viruses, in which case such infected cell will commit suicide! And 4) natural killer cells, these types of cells are responsible for destroying all cells that have been infected by a virus.

Usually, the innate immune system is good enough at controlling infections, but there are times when this system cannot cope, mainly when the amount of virus produced during the initial stages of infection is very high. It is at this time that our adaptive immune system kicks in. This system is made up of two weapons: antibodies and killer T cells (also known as CTL): The antibodies are produced in special cells known as B cells. These cells have enormous diversity of small molecular labels on its surface (cell membrane), which are used to recognize any organic molecule that may exist, such as pathogens.

When B cells find an invader (e.g. virus), a chain reaction occurs that causes many B cells to be generated that will produce only the specific antibodies required. Finally, some B cells become memory cells of the immune system; that is, they are the cells that will protect us in the event that the same invader reaches the body again. Killer T or CTL cells are white cells that, like B cells, have a wide variety of labels on their surface that are used to analyze protein fragments. Once there, they are evaluated by the CTL cells and in case of detecting an infection, the killer T cells will destroy the cell that has been infected.

However, the way in which such viruses evade these host defenses are very varied, including the production of proteins that interfere or disable the molecular alert signals of the cell (e.g. block the interferon production system), and also they can prevent the molecules involved in activating cell death programming from working; thus allowing the cell to live long enough until the virus has produced a large number of new viruses that will infect more cells. This time is exactly when Olive's phytochemical concentrates TMA+PHC should enter in action to help us.

Olive trees produce a variety of substances as product of secondary metabolism. Most of these substances participate in the defense mechanism of the plant including its virus-fighting natural phytochemical drug development whose improved evolutionary performance along the geological times allowed the very significant extension of its longevity. These metabolites, also called phytochemicals, allows also a range of uses for humans in medicine, once isolated and identified the active principles of the components with important biological activity.

The genome of the Olive trees contains genes that encode terpenoid synthase enzymes that impart terpenes with their basic structure. Such terpenoids are created by the MEP/DOXP metabolic pathway from geranyl pyrophosphate, also known as the non-mevalonic acid pathway, that occurs in the plastids (cell organelles whose main function is the production and storage of important chemical compounds used by the cell). Such Terpenes and Terpenoids are hydrocarbons with desirable properties for use in the pharmaceutical and biotechnology industries. They are the main components of the essential oils of plants that are used in traditional medicine. Vitamin A is a terpenoid.

Triterpenes are made up of three terpene units (30-carbon terpenes). This large class of molecules includes steroids and wax components from plant surfaces, such as this Maslinic acid from Olive trees or oleanolic acid from grapes. Triterpenoids are terpenes derived from triterpene molecules.

As recited below, the TMA Triterpenic compound is a metabolite from the olive trees with the ability to generate a beneficial response to them, playing a protective role against the attack of viruses and some other pathogens. Its main component, Maslinic acid is a derivative of Oleanolic acid, with which it shares structure, unlike the OH group in C-2. A characteristic common to all these terpenes —which relates their structures to biological activities—is the importance of the COO— group in C28 and the OH group in C-3. The OH group in C-2, which presents maslinic acid, and not oleanolic acid, seems to relate it to the antioxidant capacity of this compound.

It should be noted that also severe influenza is still unusual in its virulence for humans. Complications or, ultimately, death from these infections are often associated with hyper-induction of pro-inflammatory cytokine production, also known as the above mentioned “cytokine storm.” For this disease, it has been proposed that immunomodulatory therapy with TMA-PHT complex could improve outcome, with or without the combination with other modulating agents.

Regarding TMA-PHT complex activity as an antihistamine and anti-inflammatory, it has recently been published that at least one of its components Maslinic acid has in vitro activity against the enzyme isolated from the HIV-1 protease. Although this experience, we are in the process to determine in vivo that the compound is also capable of acting at the intracellular level to better defeat Covid19.

TMA-PHT components or combinations are produced already by Blue Singularity Ltd. (Gibiomed.com) as nutraceuticals and food supplements with its idroXY™ products based in TMA and/or PHT, as well as others where the composition is used to prevent and/or treat a disease or condition including oxidative stress, detoxification, inflammation, some types of cancers or related diseases or conditions, or as a nutritional supplement, and in which the composition is in the form of a tablet, a capsule, a soft gel, a liquid, a lotion, gel, powder, ointment, or spray.

Alternatively, TMA-PHT complex can be combined with other phytochemical supplement not obtained from Olive trees but mainly composed of extracts from three plants ingredients called RWS: Rosmarinus officinalis, Withania somnifera and Sophora japonica, with specific levels of carnosol/carnosic acid, with aferin A and luteolin, respectively. The synergies obtained from each ingredient TMA-PHT and RWS contributes to an important activation of the Nrf2 pathway in unique ways, leading to the up-regulation of cyto-protective human genes and a maximum protection of cells against oxidative stress.

The present invention has utility as a formulation patent that has claims also on the pharmaceutical grade drug dosage. It specifically covers two particular class of drugs, namely TMA and PHT described above, and also covers several specific formulation technologies. The patent has also claims on the chemicals and manufacturing processes used to produce the drugs. Some compounds of this patent exist in polymorphic forms with different pharmaceutical properties such as solubility and bioavailability, while the safety and efficacy profile of the active ingredients of this patent will remain similar even though the crystal form is altered. This patent covers also claims of combination of products containing two or more previously known active ingredients. Several claims of the present invention cover specific compounds and their related quantities, specially where the combination product, namely TMA-PHT complex had demonstrated a new and non-obvious synergistic effect.

In a solid composition embodiment, conventional nontoxic solid carriers may include pharmaceutical grades of cyclo-dextrines, mannitol, lactose, starch, magnesium stearate, sodium saccharine, talc, cellulose, glucose, sucrose and magnesium carbonate. Liquid pharmaceutically administrable compositions may, for example, be prepared by dissolving or dispersing an active compound with optimal pharmaceutical adjuvants in an excipient, such as water, saline, aqueous dextrose, glycerol, or ethanol, to form a solution or suspension. For example, the pharmaceutical composition may contain minor amounts of nontoxic auxiliary substances such as wetting or emulsifying agents, pH buffering agents, for example, sodium acetate or triethanolamine oleate. Actual methods of preparing such dosage forms are known, or will be apparent, to those skilled in this art.

A purified quantity of natural maslinic acid, oleanolic acid, or a combination thereof is extracted from, olive fruit and olive leaves that produce maslinic and oleanolic acids in high concentration. Crude extraction from macerated plant material of maslinic acid, oleanolic acid, or a combination thereof is defined as being substantially free of lignins, alkaloids, cellulosic material, and plant enzymes operating on maslinic acid or oleanolic acid as enzymatic substrates. It is noted that a given quantity of inventive acid is more effective upon being administered in a purified quantity relative to consumption of an equivalent inventive acid containing amount of olive fruit in combination with a pharmaceutically acceptable carrier and, in addition, may include other medicinal agents, pharmaceutical agents, carriers, or diluents.

In an oral administration embodiment, fine powders or granules may contain diluting, dispersing, or surface active agents. The fine powders or granules may be presented in water or in syrup, in capsules or sachets in the dry state, or in a nonaqueous solution or suspension. Suspending agents may also be included in tablets, which may include binders and lubricants in a suspension. Flavoring, preserving, suspending, thickening, or emulsifying agents may be also included to modify the taste and texture of the composition. The tablets and granules provided for oral administration may further be coated for ease of digestion.

Typical administrations are by oral ingestion. Ingestion is optionally with or without other food. It is further considered that variable dosing regiments are operative in the method of treatment. While in some instances a single dose treatment of TMA-PHT compound may be effective in producing therapeutic effects, in other instances a treatment period in the range of, for example, 6 weeks to 3 months may be utilized. The composition may be administered orally, parentally, or by intramuscular, injection. Injectables may be prepared in conventional forms, either liquid solutions or suspensions or solid forms suitable for solution. The dose of the dietary supplement composition may vary depending on the age, weight, general condition of the user. For example, human 65 kg male dosage is in the range of 0.1-5,000 mg of equivalent of dry maslinic acid or oleanolic acid per day may be an effective range. Preferred doses for a human 65 kg male range from 10 to 500 mg per day. Optionally, the inventive maslinic acid is present from 0.01%-5% of the dry total weight of the composition.

An inventive composition is optionally delivered to a subject daily, weekly, biweekly, monthly, or any subdivision therebetween or for longer periods. In some embodiments a subject ingests an inventive composition daily. Optionally, an inventive composition is delivered one, two, three, four, five, or more times per day.

The foregoing description is illustrative of particular embodiments of the invention, but is not meant to be a limitation upon the practice thereof. The claims, including all equivalents thereof, are intended to define the scope of the invention.

Currently it has been determined that the extraction of TMA Triterpenoid complex from the olive tree, assisted by ultrasound, especially Maslinic acid, has a high purification effectiveness compared to more conventional techniques, such as solid-liquid extraction at 40° C. and Soxtec extraction. The solvents used in the purification are also relevant, where the most known ones are ethanol or the methanol-ethanol mixture or hexane and ethyl acetate. We prefer the use of more natural and non-toxic systems without explosive potential, such as those with steam filtration and/or industrial vacuum distillation systems, and in the case of solvents, we use eutectic solvents and/or pressure extraction.

TMA component has an alpha ring hydroxyl group at the position 2 relative to one hydrogen at that position in oleanolic acid. Owing to the structural similarity therebetween, and commonality in natural sources, it has been found that maslinic acid, oleanolic acid, or a combination thereof is operative in the present invention as a treatment to fight several viruses including SARS-CoV-2 alone or in combination with PHT (Phenolic Hidroxytyrosol complex) with our TMA-PHT complex of this patent.

It is noted that other pentacyclic triterpenic acids do not exhibit the effects of TMA. As this class includes a variety of compounds identified in plant signaling, there are numerous identified pentacyclic triterpenic acids, illustratively including ursolic acid, boswelic acid, and tormatic acid.

Ways in which TMA-PHT could Help in Therapeutic Approaches Against Sars-CoV-2

Prevention: TMA-PHT could reinforce the action of antibodies to the spike protein (generated by vaccination or adoptive transfer) to block severe acute respiratory syndrome coronavirus 2 (SARS-CoV-2) from interacting with the angiotensin-converting enzyme 2 receptor (ACE2) in host cells.

Protease inhibitors against serine protease TMPRSS2 can prevent cleavage of the spike protein, which is necessary for viral fusion in the host cell. Blocking ACE2 interaction or viral fusion could prevent the virus from infecting the host cell.

CD8+T cells with virus-specific memory from a previous vaccination or infection can differentiate into effector cells during re-exposure. When they identify infected cells that have virus-specific epitopes, the infected cells are de-granulated and killed before they can produce mature virions.

This can be combined with other new treatment method that targets cytokine storm symptoms, the blood of patients with COVID-19 can be passed through custom columns that are specially designed to trap pro-inflammatory cytokines, before purified blood is passed back to patients.

Other ways of how TMA-PHT could help in additional therapeutic approaches against SARS-CoV-2 lay on its above mentioned anti-inflammatory, antiplatelet and vascular protector effects, as described below:

The effects of PHT on inflammation and platelet aggregation have been largely proven. Pre-incubation of vascular endothelial cells with PHT various antioxidant compounds such as hydroxytyrosol and oleuropein led to a decrease in monocyte adhesion to the activated endothelium and a lower production of VCAM-1. TG-rich lipoproteins isolated from individuals after ingestion of PHT, which polyphenolic compounds also reduced the production of inflammatory and vasoconstrictor eicosanoids (PGE2 and TxB2) by endothelial cells compared to those that were isolated after olive oil intake. not enriched. Another study showed that PHT could influence the lymphocyte response by inhibiting its proliferation.

On the other hand, PHT inhibited collagen-induced platelet aggregation and ADP in vitro, showing an activity comparable to potent inhibitors of platelet aggregation. It has been suggested that the mechanism by which it produces such effects could be through a selective inhibition in the synthesis of eicosanoids by the enzymes 5-lipoxygenase and 12-lipoxygenase, since a decrease in the production of TxB2 and 12-hydroxyeicosatetranoic acid by activated platelets during coagulation and a reduction in the production of LTB4 by activated leukocytes. However, HT did not affect the activity of cyclooxygenase.

PHT's natural HT has the highest known antioxidant activity, 3 times more than Coenzyme Q10, that was the leader product so far. In vitro studies have shown that it has biological properties with beneficial effects on diseases such as cardiovascular disease, neurodegenerative diseases and tumors.

Our PHT compound proved also antimicrobial properties in vitro against several infectious agents of the respiratory and the gastrointestinal tract including in vitro success trials in fighting against Haemophilus influenzae, Moraxella catarralis, Salmonella typhi, Vibrio parahaemolyticus, Vibrio cholerae or Staphylococcus aureus, even at minimum inhibitory concentrations that, in some cases, were lower to which they presented antibiotics such as Ampicillin. Recently it has been described that PHT also has activity against the causative agent of pneumonia, Mycoplasma pneumoniae and other species of the mycoplasma family. Other properties of PHT include the inhibiting from acrylamide toxicity, protection against oxidative damage produced in DNA, Hypoglycemic, and neuro-protecting action.

The antibody binding site in SARS-CoV-2 is normally not accessible to antibodies. This region is generally hidden within this virus, and is only exposed when that part of the virus changes its structure, as it would in a natural infection. Despite the slight difference between the two coronaviruses, the antibody binds much less to SARS-CoV-2 with additional Furin cleavage scissors than it does to the SARS virus, and cannot neutralize SARS-CoV-2 in laboratory tests like SARS-CoV does.

Still, the latest research findings suggest that the binding site for this antibody in SARS-CoV-2 is a site of vulnerability, and so the antibodies that bind it most closely could neutralize the virus. Such neutralizing antibodies, if converted into therapies, could be used to treat patients with COVID-19 and provide temporary protection against the virus to uninfected individuals, for example, healthcare workers.

The fact that this binding site is highly maintained among the old SARS-CoV and the new SARS-CoV-2 viruses also suggests that there may be undiscovered antibodies, that can effectively neutralize both viruses, and perhaps hopefully with the help of PMA-PHT compound we could be able to neutralize future emergencies of coronaviruses in the near future before they can cause pandemics.

One of the best described properties for PHT is its high antioxidant activity demonstrated in many trials, both in physicochemical systems, as well as in vitro biological systems and cell models. Thus, the stability of the oils to oxidation shows a clear correlation with their HT and oleuropein content. Apparently, the ortho-diphenolic structure of HT is essential so that it can exert its antioxidant activity, since other similar compounds such as tyrosol (p-hydroxy-phenylethanol), showed much less or no antioxidant activities. In addition, it has been described that thanks to the catechol ring structure, HT can also act as a metal chelator. Metal ions such as iron and copper are essential for the organism, but they can be potentially dangerous since they are able to act as electron transfers and act as catalysts for autoxidation reactions.

These antioxidant properties of PHT have a protective role against the oxidation of LDL lipoproteins and thus contribute to the prevention of cardiovascular disease. University of Granada. Facultad de Ciencias (UGR. Sc) and Visioli et al studied the effect that micro-molar amounts of HT had on the oxidation of LDL when it was incubated in vitro with CuSO4. Their results showed that HT prevented the loss of vitamin E, inhibited the formation of lipid peroxides, prevented the oxidation of PUFAs, reduced protein modification and prolonged the oxidation initiation phase of LDL lipoprotein and protected vitamin E when this lipoprotein was oxidized by CuSO4. Another study showed that pre-incubation of the plasma with an extract of the PHT caused an increase in the content of phenolic compounds in the isolated LDL and a greater resistance of it to oxidation by copper.

This activity of oxidation protection has also been reportedly described for other macromolecules such as DNA. Even at concentrations that can be considered physiological (10 HT was able to inhibit the damage caused by the H2O2 radical in the prostate cell DNA by 23%.

In cellular models it has been shown that reduced concentrations of HT could protect erythrocytes from damage caused by oxidative stress, significantly reducing hemolysis and protecting erythrocyte cell membranes from lipo-peroxidation: Thus it also reduced the oxidation of phospholipids in liver microsomes, prevented the damage caused by H2O2 and O2 in intestinal Caco-2 cells and protected against damage caused by H2O2 in neuronal cells through an increase in catalase activity.

Oxidative stress and the generation of FR (free radicals) also appear to be involved in cancer pathology and it has been suggested that PHT compounds naturally included in olive oil in very small proportions may also be related to the low incidence of various types of cancer in the Mediterranean region. Studies in cancer cells support this hypothesis. Thus, PHT was able to protect the cellular DNA of prostate cells from damage caused by FRs so it could prevent mutagenic activity caused by oxidative stress. PHT was also able to inhibit the damage caused by ultraviolet light in a melanoma cell line and PHT phenolic compounds could act directly in the colon by reducing oxidative damage. On the other hand, there could be other beneficial effects of PHT not produced by its antioxidant activity. Thus, studies with cell cultures showed that HT was able to stop the cell cycle, reducing the growth and proliferation of cancer cells, and inducing apoptosis of HL60 cells (pro-myelocytic leukemia) and HT29 cells (colon adenocarcinoma) without affecting other types of non-cancerous cells such as isolated lymphocytes and poly-morpho-nuclear cells. As disclosed above, PHT also prevents the oxidation of blood fats, prevents the formation of thrombi and improves endothelial elasticity and microcirculation.

As a result of the evolution along millions of years of the wild Mediterranean olive tree, this peculiar tree—with these sophisticated and complex molecules—managed to protect itself from the 3 main kind of threats to survive: attacks of aggressive pathogenic viruses, bacteria and fungi, as well as parasites of many kinds and the mutational induced tumors of the so-called plant cancers. The doses required for a maximum Bio-available product for humans has been determined according to our experience at BLUE SINGULARITY SL. (including formerly BIOMASLINIC SL) and our related biotech collaborating research groups.

Oxidation is the degradation of life: With the phenols of the olive trees, living beings gets help fighting oxidation. OLIVEVIR's PMA-PHT compound class of products are designed to become a solution to help prevent or minimize the occurrence of diseases of various types, including the circulatory system, avoiding infections and improving various cardiovascular aspects.

Several kinds of human-amenable phenolic compounds are present in some plants in the form of secondary metabolites that are usable by human biochemical processes. They may be involved in primary metabolism, enhance plant growth, protect the most vulnerable cells from photo-oxidation of ultraviolet light or enhance disease resistance. They are also responsible in the tree for modulating the flowering and ripening of the fruits. In the case of HT it has a fundamental role in the formation of other polyphenols.

Bioactivities of the TMA maslinic acid shown in different research results have determined several key biological properties with enormous pharmacological potential of this triterpene family, such as the above mentioned Immunomodulatory effect, Antimicrobial capacity by serine protease inhibition, analgesic, hepatic-protective, antitumor, anti-inflammatory, antioxidant, Neuroprotective effect, immune system effector properties, as follows: Anti-proliferative effect, Prevents oxidative stress, Effect on enzymatic activities, Inhibition cholesterol acyl transferase, Glycogen phosphorylase inhibition, Inhibition of topoisomerase DNA and DNA polymerase, Elastase inhibition, Lipid lowering effect.

Concerning the in vitro assays of TMA with other viruses: In the MT2 cell line assays, Maslinic acid at concentrations of 25 and 30 μg/ml was able to inhibit replication of a primary HIV-1 isolated. At 25 μg/ml decrease was detected of the cytopathic effect and the level of p24 antigen in the culture supernatant, and at 30 μg/ml absence total cytopathic effect and also decrease in p24 antigen.

The total destruction of the viral culture was observed from concentrations of 25 μg/ml of TMA's Maslinic acid, while between 5 and 20 μg/ml, a decrease in viability was observed between 57% and 19%. The Maslinic acid concentrations between 5 and 20 μg/ml did not produce any inhibitory effect on the replication in none of the cultures infected with different amounts of HIV-1.

These results, good enough and without manifesting toxicity at therapeutic doses, constitute the basis for the preparation of pharmaceutical formulations containing OLIVEVIR's TMA-PHT complex or its compounds TMA or PHT, alone or in combination with other materials that complement or synergize their action, as active matter against viruses like the HIV infection (that killed 40 million people worldwide since 1984) in an amount that can go up to 2500 mg/day of MA acid if necessary from TMA for 2/3 weeks.

To increase the solubility and bioavailability of TMA (the entire Triterpenic compound), we use Cyclodextrins, mainly those of the Hydroxypropyl-β-cyclodextrin type, which are the ones that allow the highest bioavailability (up to approx. 60%) to make the inclusion complexes, although, instead, simple virgin olive oil can be used to dilute to a lesser extent the TMA, so its bioavailability can increase to a total of approx. 20%. Other synergistic technics to increase bioavailability such as Micro-sizing and Nano-formulation are also used. On the other hand, DMSO (dimethyl sulfoxide) has also been used to a lesser extent on some other occasions as a solubilizing agent for TMA.

Cyclodextrins (CDs) are cyclic oligomers consisting of six or more units of α-D-glucopyranose linked by 1-4 bonds. They are obtained by a degradation reaction of the cyclodextrin-glucotransferase enzyme on starch. Both natural CDs and their different derivatives can form inclusion compounds with a wide variety of biochemical products like TMA, modifying the physicochemical properties advantageously. Cyclodextrins (CDs) belong to the family of molecules whose structure is made up of a dimensionally stable hydrophobic cavity that can trap or encapsulate other molecules. The remarkable encapsulation properties lead to a “host-host” type relationship that can be modified and/or improved in the physical and chemical properties of the host molecule.

Among the most advantageous modifications are reduced toxicity, increased solubility, intrinsic, improved selectivity in chemical separation/catalysis process, formation of polymers of CDs, etc. One of the most widely used derivatives is hydroxypropyl CDs, which have a high solubility and low toxicity with respect to source CDs, which makes them an ideal candidate for vehicle of lipo-active ingredients of low solubility such as our TMA solutions. With different degrees of substitution, hydroxypropyl b-CD stands out, one of the CDs that resulted more recommendable in our case.

The term inclusion compound is also known through other names, such as adduct, clathrate, molecular and complex compound. We apply different aspects of the complexation of TMA-PHT with CDs, to increase its solubility and bioavailability, allowing the incorporation of a higher concentration of drugs in the TMA-PHT complex or any combination of its ingredients in the same oral solution in different amounts for the different diseases to be treated including Covid19.

Interactions between TMA and a series of CDs, specifically β-CD and hydroxypropyl β-CD (HP-b-CD) were studied when they form inclusion compounds through molecular modeling. For the correct performance of this technique it was necessary to start from the lowest energy monomers (drugs and CDs). The results obtained suggest the formation of stable complexes and therefore viable, for which hydrogen bridges were observed to play a very important role, also confirming the complex stability.

The most recommended CDs for the TMA-PHT or TMA alone is Hydroxypropyl-β-cyclodextrin while depending on whether we do the inclusion polymer complex preparation in a liquid or solid medium, the most common inclusion Ciclodextrine+TMA compound elaboration methods are:

In liquid medium (Preparation in solution). It can be prepared by any of the different methods below described or a combination of them:

Co-precipitation: The mixture is stirred at constant temperature until equilibrium is reached. The complex precipitates as a microcrystalline powder, is filtered and dried.

Neutralization: It consists of adding to an acid or basic solution of CD, the active principle in the form of salt, the pH of the medium is adjusted to neutrality and the precipitate obtained is filtered and subjected to drying (Moyano et al., 1998).

Atomization: It is based on preparing a solution of the CD and the active ingredient in water or organic solvent, it is subjected to agitation during, after which it is introduced into an atomizer (Spray-Dryer), where it is nebulized as droplets at high temperature, the solvent evaporating and collecting the solute particles.

Lyophilization: The procedure is the same as in the previous method, but in this case the solution is lyophilized, isolating the inclusion compound. —Evaporation (co-evaporation): Consists of preparing a solution, generally ethanolic, of the CD and of the active ingredient in whole molar proportions (1:1, 1:2 3:4), it is subjected to stirring and the solvent.

Preparation of TMA-PHT complexes in solid medium by any of the different methods below described:

Kneading: it is used in drugs of low solubility. The mixture of active ingredient and CD together with an aqueous or hydroalcoholic solution is kneaded in a mortar and the mass is subsequently dried until complete evaporation of the liquid.

Crushing: Consists of crushing the mixture of CD and active ingredient using different techniques (vibrational mill, ceramic mill, mortar, etc.).

Fusion: By fusion of the active principle, to which the pulverized CD is added. After cooling, the excess is removed by washing with a solvent that has a low tendency to be included in the CD cavity.

Heating in sealed container: It consists of introducing a crushed mixture of CD and active ingredient in a closed container, it is subjected to stirring and heating to a defined temperature and time.

Compaction method: It is based on subjecting a physical mixture of drug and CD to the action of a hydraulic press, leading to the formation of the inclusion compound.

The simplest way to make our inclusion complexes is to put the host molecule in aqueous solution. However, it should be noted that inclusion complexes that are formed in this way result a simplified version of a process that is much more complex. CDs are modified by forming inclusion complexes with host molecules as non-inclusion complexes. Hydroxyl groups present on the outer surface of the CD can form hydrogen bonds with other molecules. Another possibility is the formation of mobile aggregates to dissolve lipophilic molecules insoluble in water. In recent years, in the pharmaceutical industry, it has been seen that other types of complexes, such as non-inclusion complexes, also participate in the solubilization of drugs. The formation of these aggregates between CD molecules and drugs can increase their bioavailability. CD aggregates have the ability to form complexes, nano-aggregates, and nanotubes with host/host interactions.

Further in this invention, as said above, we include the use of other techniques to overcome the problem of TMA bioavailability, including the use of Micro-sizing and Nano-formulations to allow TMA medication to become effectively absorbable and bioavailable. Nano-formulations reduce the size of the nutrient or medication particles down to tiny micron levels and sometimes attach the nutrient to a second component (like the above mentioned CDs and/or olive oil) that allows for the particle to be much better absorbed. Studies have demonstrated that nano-formulations such as TMA improved absorption as much as 30 times more effective.

In accordance with one embodiment, the invention includes a composition consisting of TMA and/or PHT with two or more olive phytochemicals being present in the composition in an amount effective to activate the effect of the TMA-PHT several different therapeutic potentials of these drug(s) on each particular medical target(s) and its desired results, including its effect as an inhibitor of protease activity and/or activating the Nrf2 (Nuclear factor-erythroid 2 related factor 2) pathway, or other medical targets or desired effect(s). This claim is referred to the use of this Triterpenic phytochemical lipophilic compound TMA extracted from various parts of the olive tree (mainly fruits, leaves and seeds) and subsequently concentrated, which includes, in addition to Maslinic acid (the most abundant triterpene in olive trees, ranging from 20% to 70% and typically more than 40% vol. of all TMA fraction, Oleanolic acid (ranging 10-40%) and small concentrations or traces of other natural components such as Erythrodiol, Beta-amirin, Beta-amirone, as well as triterpenes derived from Lupane (lupeol, lupone), derivatives of Ursan (Ursolic acid, Uvaol) and derivatives of Taraxane (Taraxerol) completing the remaining Triterpenic fraction, on one hand, and on the other hand, the Phenolic compound PHT extracted from olive fruits that includes 160 polyphenols (with a separate and different extraction and concentration technology procedure), which includes Hydroxytyrosol (with more that 40% and ranging from 20%-60%), Oleuropein (2% to 30%) and smaller amounts of minor olive polyphenols including Tyrosol (1 to 10%), Oleocantal, Oleacein, Hydroxypheniletanol, Quercetine, Luteoline, Rutine, Verbacosid, Floridzine, as well as Galic, Clorogenic, Vanilic, Cafeic, Cumaric and Transcinamic acids, and very small or even trace amounts of other olive polyphenols totaling completing the remaining olive phenolic fraction, and/or the mixture of both groups of compounds TMA and PHT hereinafter TMA-PHT Complex, called OLIVEVIR in any proportions, including the typical ones ranging from 25% to 75% TMA and 75% to 25% PHT (depending on the target or desired use of the medication) or any of its components or combinations, as drugs and/or pharmaceutical products for the preparation, alone or in combination with other agents, of medicines for the treatment of human or animal diseases caused by any agent, including agents that cause COVID19, which is characterized in that the product has several medical effects including the effect as an inhibitor of protease activity.

The composition may include the use of TMA-PHT or any of its components as enzyme inhibitors, alone or in combination with other compatible phytochemicals and other suitable agents, for the preparation of drugs to be used in amounts effective for the prevention and/or the treatment of COVID19 or any other human or animal disease according to the first claim in any type of pharmaceutical drug form, in any form of Galenic administration and at any dose per individual and day.

The composition may include the use of the triterpenic compound mainly based on maslinic acid and the polyphenolic compound mainly based on hydroxytyrosol (all hereinafter TMA-PHT complex or OLIVEVIR), as pharmaceutical drugs in an amount effective to prevent or treat each infection or disease, as well as the methods, elements, combinations, doses and adjuvant products to improve its solubility, bioavailability and other characteristics for the treatment of Covid19 and/or other diseases or conditions through its administration thereof. Such typical administrations are by oral ingestion which is optionally with or without other food. It is further considered in this claim that variable dosing regiments are operative in the method of treatment, so while in some instances a single dose treatment of TMA-PHT compound may be effective in producing therapeutic effects, in other instances a treatment period in the range of, for example, 6 weeks to 3 months may be utilized. The composition may be administered orally, which is the most common or parenterally, where injectables may be prepared in conventional forms, either liquid solutions or suspensions. Preferred doses include 600 mg hard capsules, each containing 200 mg olive oil, 200 mg PHT, 200 mg TMA, with a typical dose of 3 capsules a day or alternatively the gastro-resistant capsules of 500 mg, 250 mg cyclodextrins 250 mg TMA, enhancing Maslinic acid bioavailability drastically to 60%. and separated soft-gels of PHT, whose bioavailability is very high and needs no adjuvant. Depending on the case, the disease and hence the treatment these inventive compositions can be optionally delivered in an effective amount to a subject daily, weekly, biweekly, monthly, or any subdivision therebetween or for longer periods. In some embodiments a subject ingests an inventive composition daily. Optionally, an inventive composition is delivered one, two, three, four, five, or more times per day.

The composition may include the use of the TMA-PHT complex as an active medical element in therapeutic objectives related to COVID19 disease, including human glandular epithelia of the respiratory and digestive tracts and endothelia, including the use of this compound TMA-PHT, as a serine protease inhibitor in an amount effective for inhibit such serine protease in the treatment of any viral disease or infection, including this Covid19 disease caused by SARS-CoV-2.

The composition may include the use of the TMA-PHT complex, with the said triterpenic compound (based on maslinic, oleanoic acid and other components of the lipophilic fraction of the Olive) along with the best and strongest natural anti-oxidant known in nature, with the highest anti-oxidant capacity: This PHT polyphenolic compound (based on hydroxytyrosol, oleuropein and 158 minor or trace polyphenols) as drug(s) or pharmaceutical product(s), in any combination of its components for the treatment of diseases characterized by being applicable on the basis related to its properties and effects, including anticancer, anti-proliferative, antioxidant, immunomodulatory, antimicrobial, neuroprotective, lipid-lowering and vaso-modulatory effects, as well as inhibitor of cholesterol, acid-transferase, glycogen phosphorylase, topoisomerase, DNA polymerase and elastase, and also preventive of oxidative stress, and as antiviral medicine, with suitable compositions and methods for preventing or treating certain health conditions in an amount effective for activating each of these properties including the ones associated with factors and pathways inhibiting inflammation or oxidative stress.

The composition may include the use of the TMA-PHT complex, or simply its TMA component or other of its components as an anti-inflammatory drug and a method to act selectively (among others) on Interleukin IL6, in an amount of TMA effective for inhibiting it, and the use of this drug also as a strong immuno-modulator tool. These compositions are prepared from ingredients containing such TMA-PHT complex of phytochemicals from Olive trees and other sources that also activates the Nrf2 pathways, all improved by the several synergistic effects among these active principles and the potent anti-inflammatory capacity of the TMA-PHT complex of this invention since it also inhibits the formation of COX2 and also inhibits some pro-inflammatory interleukins, as recited in the following claim No 7.

The composition may include the use of the TMA-PHT complex, or simply its TMA component or other of its components for specific actions respectively in relation to inhibiting COX-2 (enzyme that accelerates the formation of substances that cause inflammation), prostaglandins (mediators that play an important role in the regulation of the allergic inflammatory response), and/or TNF-alpha biomarker (biomarker of the inflammatory pathway). Such COX-2 inhibitors of this claim are a type of nonsteroidal anti-inflammatory drug (NSAID) that directly targets cyclooxygenase-2. COX-2 also appears to be related to cancers and abnormal growths in the intestinal tract. So, COX inhibitors have been shown to reduce the occurrence of cancers and pre-cancerous growths, as well as neurodegenerative diseases. And as far as the efficacy of corticosteroids in viral ARDS remains very controversial, non-steroidal TMA-PHT anti-inflammatory drug (NSAID) of this claim can be used alone or combined with other steroids including Dexametasone, that proved effectiveness in 33% of cases of mechanically ventilated adult patients with established moderate-to-severe ARDS caused by confirmed Covid-19 infection, admitted in a network of Spanish ICUs. The acute respiratory distress syndrome (ARDS) is a catastrophic illness of multifactorial etiology characterized by a diffuse, severe inflammatory process of the lung leading to acute hypoxemic respiratory failure requiring mechanical ventilation (MV). Clinical and experimental research has established a strong association between dysregulated systemic and pulmonary inflammation and progression or delayed resolution of ARDS.

The composition may include the use of the TMA-PHT complex, or simply its TMA component or other of its components is made as a preventive medical element (s) in an amount effective for a significant prevention rate for any specific disease, including Covid19 viral disease, (also taking advantage of the very low risk of rejection by the patient due to the low-zero toxicity of this OLIVEVIR's TMA-PHT complex), as well as the method of claim 1, where in the total TMA and/or PHT or any of the components is used for its immuno-modulator effect and/or treatment in an amount effective to fight human or animal cytokine storms or sHLH. The composition TMA-PHT of this claim can be used also as an important cellular regenerator at low doses and at high doses as a potent selective inducer of apoptosis in cancer cells. Additionally, according to this claim, this composition can be used taking advantage of its notable activity in the correction of GEN P53, as a guardian of the genome. P53 is the gene that acts to eliminate mutated cells, so that when P53 is altered, the body has no endogenous defense to fight against carcinogenic processes.

The composition may include the use of TMA and/or PHT or other of its components according to claim number 1 as a direct or indirect treatment for any infection, including Covid19, and including the use of TMA and/or PHT in an amount effective for the prevention or treatment of inflammation, especially critical during cytokine storm episodes in humans. Taking into account that every place where any viral protein like the SARS-CoV-2 viral ones interacts with a human protein it represents a potential pharmacological site, therefore the various elements of this TMA-PHT drug cocktail could act on different objectives. This claim includes the use of TMA-PHT complex in any preparation to improve the gut microbiota enhancing the human immune system. It is well documented that such gut microbiota has a critical role in the immune system and therefore its composition could affect vulnerability and disease outcomes, including the ones of COVID-19.

The composition may include the use of TMA-PHT for the treatment and/or prevention of thrombosis. The fact that coagulopathy occurs in patients with COVID-19 and other diseases has promoted anti-thrombotic strategies. This claim includes the use of TMA-PHT complex in any preparation, including the PHT compound of this claim, alone or in combination with low molecular weight heparins (LMWH) or other anticoagulant in an effective amount that could be recommended when D-dimer values are higher than normal. Therapeutic anticoagulation is normally reserved for cases in which a clear local or systemic thrombotic pathology is seen or foreseen.

The composition may include the use of TMA-PHT—or any of its components as an antibiotic drug for any infections including COVID19 and/or antiproliferative and/or anti-inflammatory drug to obtain a reduction in the generation of pro-inflammatory cytokines in any disease including COVID19, as it has been proven in our trials to inhibit the spread of the HIV virus by inhibiting replication of a primary HIV-1 isolated as well as decreased cytopathic effect and p24 antigen levels in MT2 cells and attenuated intracellular oxidative stress by inhibiting the production of NO and H2O2. It is claimed hereby the composition wherein the use of TMA-PHT or its component TMA Triterpenic complex as an antibiotic, taking advantage of the antimicrobial properties, especially the ones of TMA's Oleanolic and Maslinic acids, and such strong antimicrobial activity has been tested in vitro: After synthesizing and studying fourteen oleanolic and maslinic acid derivatives, the research team found two, called OA-HDA and MA-HDA, that exceeded the antimicrobial activity of the original compounds both in vitro and in an animal model. And most importantly: the bacteria did not demonstrate the ability to generate resistance to either compound. This is relevant now when the inappropriate use of antibiotics has resulted in the emergence of bacteria resistant to multiple drugs, that are capable of surviving despite the use of such drugs, like in the case of Nosocomial hospital infections: About 5% of all patients acquire an infection during their hospital stay. They are generally caused by the Staphylococcus aureus bacteria and are associated with increased morbidity and mortality, as well as a great economic impact on the health system. Most nosocomial infections originate during invasive procedures, such as the application of catheters or prostheses. Unfortunately, the surfaces of these devices favor the formation of biofilms, bacterial ecosystems with rates of resistance to antibiotics up to 1000 times higher than normal bacteria. The only effective treatment against a biofilm infection is medical device removal and long-term antibiotic therapy, which can promote antibiotic-resistant bacteria. In such a situation, the compounds of TMA-PHT of this claim are very useful, since their antimicrobial activity in the biofilms of the catheter has also been tested, with positive results and act effectively without the danger of causing resistance to antibiotics.

The composition may include the use of TMA-PHT—or any of its components like MA—as an immuno-modulator and to improve endothelial function, avoiding lipid peroxidation and suppressing the generation of superoxide anions, taking advantage of the fact that these penta-cyclic Triterpenes of this invention possess antioxidant properties, and Maslinic acid in particular exhibits properties pro and anti-inflammatory depending on its chemical structure and its dose, and may be useful to modulate the immune response due to the immune-modulatory behavior of this triterpene. TMA acts selectively on Interleukin IL6, inhibiting it, thus interfering with the JAK-STAT signaling pathway. This inhibitor has therapeutic application in an amount effective in the treatment of inflammatory diseases with an important immuno-modulating effect and so, it could be used alone or combined with other JAK inhibitors that already reached clinical trials like Tofacitinib.

The composition may include the use of the TMA-PHT complex, or any of its components according to claims 1 to 12, in any type of pharmaceutical form, in any Galenic administration form and at any dose per individual and day, and the process for preventing or treating a disease or medical condition in a subject in need thereof comprising: administering to said subject a composition comprising purified amounts of TMA, or said purified amounts mixed with PHT; wherein said TMA compound comprises 20%-80% of the dry weight of the composition. This claim includes its use for prevention of COVID 19, according to Claim n° 8, as far as TMA-PHT complex may enhance the action of the antibodies against the spike protein (generated by vaccination or adoptive transfer) to prevent the severe acute respiratory syndrome coronavirus 2 (SARS-CoV-2) from interacting with the receptor for the converting enzyme of angiotensin 2 (ACE2) in host cells. This claim also includes the use of TMA-PHT complex combined with other phytochemical supplement not obtained from Olive trees, including the ones composed of extracts from three plants ingredients called RWS: Rosmarinus officinalis, Withania somnifera and Sophora japonica, with specific levels of carnosol/carnosic acid, with aferin A and luteolin, respectively. The synergies obtained from each ingredient TMA-PHT and RWS contributes to an important activation of the Nrf2 pathway in unique ways, leading to the up-regulation of cyto-protective human genes and a maximum protection of cells against oxidative stress.

The composition may include the use of TMA-PHT complex, and/or any of its components or combinations according to claims 1 to 13 as a Nutraceutical products or food supplements for humans or animals. This claim includes the activity of TMA-PHT complex of this invention or any of its components in the dermo-cosmetics area, that does not have any current formulation with these ingredients worldwide. So, we are pioneering and herby claiming its usage in dermo-cosmetic lotions and related dermic health applications. This claim includes—as well—the composition(s) comprising these Olive tree phytochemicals that could also include phytochemicals selected from other natural sources including carnosol, carnosic acid, shogaol, gingerol, luteolin, withaferin A, and/or other phytochemical(s) of this kind being present in the composition in an amount effective to activate the Nrf2 (Nuclear factor-erythroid 2 related factor 2) pathway, to fight oxidative stress.

A method for obtaining and preparing TMA and PHT, as described above, consisting primarily of a mixture of a physiologically acceptable mixture of TMA and PHT phytochemicals, all of which include the steps of the extraction of TMA from fruits, seeds and leaves of olive, and/or products obtained in a manufacturing process of olive oil, or mixtures thereof, with water, a hydrophilic organic solvent or mixtures thereof, and then concentrating the resulting TMA extract on one hand and in the other hand: a method for obtaining and preparing PHT consisting of a mixture of polyphenols, mainly Hydroxytyrosol and Oleuropein in a physiologically acceptable mixture thereof, all of which includes the steps of their extraction from plants of olive, or products obtained in a manufacturing process of olive oil, or mixtures thereof, with water, an organic solvent or mixtures thereof, and then concentrating the resulting extract, and then carrying out a purification treatment by fractionation of the resulting extract. The extraction of TMA Triterpenoid complex from the olive tree claimed here can be assisted by ultrasound, especially Maslinic acid, that has a high purification effectiveness compared to more conventional techniques, such as solid-liquid extraction at 40° C. and Soxtec extraction. The solvents used in the purification are also relevant, where the most known ones are ethanol or the methanol-ethanol mixture or hexane and ethyl acetate. We claim hereby the use of more natural and non-toxic systems without explosive potential, such as those with steam filtration and/or industrial vacuum distillation systems, and in the case of solvents, we use eutectic solvents and/or pressure extraction.

The method may include the step of treating the resulting extracted substance or liquid olive extract with a basic medium, a basic substance, or both, and the method of claim 15 optionally further comprising the step of: treating the resulting extracted substance or the extracted liquid with an acidic medium, an acidic substance, or both, as well as the method of claim 15 wherein the TMA and PHT products obtained in the olive oil manufacturing process from the olive plant (fruits, seeds, bark, leaves, stems, and olive sprouts or germs) and dry products, powdered products and defatted products of these ingredients, comprise at least one member selected from the group consisting of compressed residues, extraction residues, squeezed oil, extracted oil, foam of degummed oil, de-acidified foam oil, dark oil, agent waste bleach, deodorized foam, juice expressed in oil, sewage, and waste filter media. The present invention has utility as a formulation patent that has this claim also on the pharmaceutical grade drug dosage. It specifically covers two particular class of drugs, namely TMA and PHT described above, and also covers several specific formulation technologies and hereby claims on the chemicals and manufacturing processes used to produce the drugs. Some compounds of this patent application exist in polymorphic forms with different pharmaceutical properties such as solubility and bioavailability, while the safety and efficacy profile of the active ingredients of this patent application will remain similar even though the form is altered. This claim covers also the combination of products containing two or more previously known active ingredients, specific compounds and their related quantities, specially where the combination product, namely TMA-PHT complex had demonstrated a new and non-obvious synergistic effect. In a solid composition embodiment of this claim, conventional nontoxic solid carriers may include pharmaceutical grades of cyclo-dextrines, mannitol, lactose, starch, magnesium stearate, sodium saccharine, talc, cellulose, glucose, sucrose and magnesium carbonate. Liquid pharmaceutically administrable compositions may, for example, be prepared by dissolving or dispersing an active compound with optimal pharmaceutical adjuvants in an excipient, such as water, saline, aqueous dextrose, glycerol, or ethanol, to form a solution or suspension. For example, the pharmaceutical composition may contain minor amounts of nontoxic auxiliary substances such as wetting or emulsifying agents, pH buffering agents, for example, sodium acetate or triethanolamine oleate. A purified quantity of natural maslinic acid, oleanolic acid, or a combination thereof is extracted from, olive fruit and olive leaves that produce maslinic and oleanolic acids in high concentration. Crude extraction from macerated plant material of maslinic acid, oleanolic acid, or a combination thereof is defined as being substantially free of lignins, alkaloids, cellulosic material, and plant enzymes operating on maslinic acid or oleanolic acid as enzymatic substrates. It is claimed that a given quantity of inventive is more effective upon being administered in a purified quantity relative to consumption of an equivalent quantity containing amount of olive fruit in combination with a pharmaceutically acceptable carrier and, in addition, may include other medicinal agents, pharmaceutical agents, carriers, or diluents.

The method may include an embodiment in which the residues of the olive oil production process result in phytochemicals, the extraction of which is carried out using water, a hydrophilic organic solvent or mixtures thereof or where the extraction is carried out under pressure at which the precipitates formed by cooling are recovered and at which the total content of oleanolic acid, maslinic acid and physiologically acceptable products thereof, present in a mixture comprising oleanolic and maslinic acid prepared according to the fractionation treatment—purification consisting of recrystallization, re-precipitation, purification by normal phase chromatography, purification by reverse phase chromatography, purification by normal phase and reverse phase chromatography, decolorization and deodorization. The resulting drug capsules will have a TMA to PHT ratio of around 50%-50% for antiviral purposes, however the TMA to PHT ratio for other fines could range from 30%-70% to 70%-30%, depending on the medical or nutraceutical objective, and the TMA could be integrated into inclusion complexes with cyclodextrins in a ratio of 50%-50% TMA/CD in hard capsules typically 500 mg, or 33%-33%-33% of virgin olive oil/TMA/PHT in the case of the soft gel capsule, typically 600 mg, in any type of pharmaceutical form, in any form of galenic administration and in any dose per individual and day.

In accordance with one embodiment, a method for treating and/or preventing a disease or condition includes the step of administering a composition to humans or animals, the composition comprising one or more phytochemicals selected from the group of Olive Terpenes (TMA) and/or Polyphenols (PHT) and combinations thereof and that may include phytochemicals from other plants to reinforce the amounts of very minor phytochemicals in the olive, including carnosol, carnosic acid, shogaol, gingerol, luteolin and/or conferin A in an amount effective to activate the Nrf2 pathway (NF-E2 related factor 2), the composition comprises at least one other phytochemical ingredient selected from the group consisting of rosemary, ginger, ashwagandha, milk thistle and bacopa. Also rosemary extract (ursolic acid), ginger extract in addition to milk thistle extract and Bacopa monnieri extract, being approximately the ratio between said rosemary extract, ginger extract, luteolin, milk thistle extract and Bacopa monnieri extract.

In one embodiment, the TMA-PHT complex or the TMA compounds are made more soluble and bioavailable using one or any combination of the below described methods including the preparation in cyclo-dextrines (CD's) or inclusion complexes to increase the solubility and bioavailability of the Triterpenic complex TMA. In this invention we claim the use of the Hydroxypropyl-β-cyclodextrin type, which are the ones that allow the highest bioavailability (up to approx. 60%) to make the inclusion complexes, and/or using simple virgin olive oil to dilute the TMA, so the bioavailability of its MA can be increased to 20%. Among the most advantageous modifications are reduced toxicity, increased solubility, intrinsic, improved selectivity in chemical separation/catalysis process, formation of polymers of CDs, etc. The hydroxypropyl CDs used in this invention shows a higher solubility and lower toxicity with respect to other CDs, which results in making them an ideal candidate as a vehicle for this lipo-active ingredients of low-solubility Maslinic acid rich solutions. With different degrees of substitution, hydroxypropyl b-CD allows the incorporation of a higher concentration of drugs in the TMA-PHT compound or in any combination of its ingredients in the same oral solution in different amounts as required for the different diseases to be treated or for COVID-19 treatment. Interactions between TMA and a series of CDs, specifically β-CD and hydroxypropyl β-CD (HP-b-CD) were studied when they form inclusion compounds through molecular modeling. For the correct performance of this technique, it was necessary to start from the lowest energy monomers (drugs and CDs). The results obtained suggest the formation of stable complexes and therefore viable, for which hydrogen bridges were observed to play a very important role, also confirming the complex stability. Additionally, as mentioned above in this claim of this invention we also include the use of some other techniques to overcome the problem of bioavailability, including the use of Micro-sizing and Nano-formulations to allow TMA medication to become effectively absorbable and bioavailable. Nano-formulations reduce the size of the nutrient or medication particles down to tiny micron levels and sometimes attach the nutrient to a second component (like the above-mentioned CDs and/or olive oil) that allows for the particle to be much better absorbed. Studies have demonstrated that nano-formulations such as TMA improved absorption as much as 30 times more effective, and as a third alternative, DMSO (dimethyl sulfoxide) can also be used for solubilizing MA.

In accordance with one embodiment, the invention includes the application and use of the phytochemical complex TMA-PHT (OLIVEVIR), including its components or combinations according to claims 1 to 19 is quite diverse. Regarding the COVID19, it is claimed hereby its use as preventive of this disease, as well as its usage during the three phases of COVID19 disease with its different therapeutical targets as described in this patent and also its use in post-COVID stage treatments, when required, including PPCS (persistent post-COVID-19 syndrome) cases. TMA-PHT (OLIVEVIR) can be used effectively in diverse forms as a nutraceutical or food supplements and/or drugs to be used as a preventive of various diseases, as well as the treatment of the disease itself in case of infection and/or the treatment of the consequences of this disease, such as post-COVID sequelae treatment, as recited above, using these active elements of TMA-PHT to treat PPCS (persistent post-COVID-19 syndrome), as it is claimed hereby. These drugs also have applications for various other diseases and conditions for humans and animals, such as TMA-based medical lotions for treating superficial arthritic diseases in places like knees and elbows and also different forms of use, where it is used in an effective amount. to prevent and/or treat each applicable disease or condition, including oxidative stress, detoxification, inflammation, some types of cancer, or a related disease or condition and wherein the composition is in the form of a tablet, capsule, gel smooth, liquid, lotion, gel, powder, ointment, or spray, hereby claimed.

Those skilled in the art will appreciate that embodiments of the present disclosure may be practiced in conjunction with any number of systems, and that the systems described herein are merely exemplary embodiments of the present disclosure. Further, the connecting lines shown in the various figures contained herein are intended to represent example functional relationships and/or physical couplings between the various elements. It should be noted that many alternative or additional functional relationships or physical connections may be present in an embodiment of the present disclosure.

As used herein, the terms “module” or “controller” refer to any hardware, software, firmware, electronic control component, processing logic, and/or processor device, individually or in any combination, including without limitation: application specific integrated circuits (ASICs), field-programmable gate-arrays (FPGAs), dedicated neural network devices (e.g., Google Tensor Processing Units), electronic circuits, processors (shared, dedicated, or group) configured to execute one or more software or firmware programs, a combinational logic circuit, and/or other suitable components that provide the described functionality.

As used herein, the word “exemplary” means “serving as an example, instance, or illustration.” Any implementation described herein as “exemplary” is not necessarily to be construed as preferred or advantageous over other implementations, nor is it intended to be construed as a model that must be literally duplicated.

While the foregoing detailed description will provide those skilled in the art with a convenient road map for implementing various embodiments of the invention, it should be appreciated that the particular embodiments described above are only examples, and are not intended to limit the scope, applicability, or configuration of the invention in any way. To the contrary, various changes may be made in the function and arrangement of elements described without departing from the scope of the invention.

### Abbreviations Used

ACE: Angiotensine converting enzyme;
ARDS: Acute respiratory distress syndrome;
CD: Cyclodextrines;
DC: Dextrine complexes;
DMSO: dimethyl sulfoxide;
EFSA: European Food Safety Agency;
FDA: Food and Drug Administration (USFDA);
FR: Free radicals;
HT: Hydroxytyrosol;
HMA: hydrolyzed maslinic acid;
ICU: Intensive care unit;
IL: Interleukine;
JAK: Janus kinase enzyme;
LDL: Low density lipoproteins (“bad” cholesterol);
LMWH: Low molecular weight heparin;
MA: Maslinic acid;
MDCK: Madin-Darby Canine Kidney cells;
NO: Nitric Oxide;
NSAID: Nonsteroidal anti-inflammatory drug;
OA: Oleanoic Acid;
OE: Oleuropeine;
OO: Olive Oil;
PAI: Plasminogen activator inhibitor;
PGE: Prostaglandine;
PHT: Polyphenolic compound (mainly from Olive trees);
PUFA: Polyunsaturated fatty acid;
sHLH: Secondary haemophagocytic lympho-histiocytosis;
TMA: Triterpenoid compound (mainly from Olive trees);
TMA-PHT complex: Mix of TMA and PHT, at 50% Vol.; and
TMPRSS: Transmembrane protease serine.

1. A composition consisting of TMA and/or PHT with two or more olive phytochemicals being present in the composition in an amount effective to activate the effect of the TMA-PHT several different therapeutic potentials of these drug(s) on each particular medical target(s) and its desired results, including its effect as an inhibitor of protease activity and/or activating the Nrf2 (Nuclear factor-erythroid 2 related factor 2) pathway, or other medical targets or desired effect(s).

2. The composition of claim 1, wherein the use of TMA-PHT or any of its components as enzyme inhibitors, alone or in combination with other compatible phytochemicals and other suitable agents, for the preparation of drugs to be used in amounts effective for the prevention and/or the treatment of COVID19 or any other human or animal disease according to the first claim in any type of pharmaceutical drug form, in any form of Galenic administration and at any dose per individual and day.

3. The composition of claim 1, wherein the use of the triterpenic compound is substantially based on maslinic acid and the polyphenolic compound mainly based on hydroxytyrosol (all hereinafter TMA-PHT complex or OLIVEVIR), as pharmaceutical drugs in an amount effective to prevent or treat each infection or disease, as well as the methods, elements, combinations, doses and adjuvant products to improve its solubility, bioavailability and other characteristics for the treatment of Covid19 and/or other diseases or conditions through its administration thereof.

4. The composition of claim 1, wherein the use of the TMA-PHT complex as an active medical element in therapeutic objectives related to COVID19 disease, including human glandular epithelia of the respiratory and digestive tracts and endothelia, including the use of this compound TMA-PHT, as a serine protease inhibitor in an amount effective for inhibit such serine protease in the treatment of any viral disease or infection, including this Covid19 disease caused by SARS-CoV-2.

5. The composition of claim 1, wherein the use of the TMA-PHT complex, with the said triterpenic compound (based on maslinic, oleanoic acid and other components of the lipophilic fraction of the Olive) along with the best and strongest natural anti-oxidant known in nature, with the highest anti-oxidant capacity: This PHT polyphenolic compound (based on hydroxytyrosol, oleuropein and 158 minor or trace polyphenols) as drug(s) or pharmaceutical product(s), in any combination of its components for the treatment of diseases characterized by being applicable on the basis related to its properties and effects, including anticancer, anti-proliferative, antioxidant, immunomodulatory, antimicrobial, neuroprotective, lipid-lowering and vaso-modulatory effects, as well as inhibitor of cholesterol, acid-transferase, glycogen phosphorylase, topoisomerase, DNA polymerase and elastase, and also preventive of oxidative stress, and as antiviral medicine, with suitable compositions and methods for preventing or treating certain health conditions in an amount effective for activating each of these properties including the ones associated with factors and pathways inhibiting inflammation or oxidative stress.

6. The composition of claim 1, wherein the use of the TMA-PHT complex, or simply its TMA component or other of its components as an anti-inflammatory drug and a method to act selectively (among others) on Interleukin IL6, in an amount of TMA effective for inhibiting it, and the use of this drug also as a strong immuno-modulator tool.

7. The composition of claim 1, wherein the use of the TMA-PHT complex, or simply its TMA component or other of its components for specific actions respectively in relation to inhibiting COX-2 (enzyme that accelerates the formation of substances that cause inflammation), prostaglandins (mediators that play an important role in the regulation of the allergic inflammatory response), and/or TNF-alpha biomarker (biomarker of the inflammatory pathway). Such COX-2 inhibitors of this claim are a type of nonsteroidal anti-inflammatory drug (NSAID) that directly targets cyclooxygenase-2. COX-2 also appears to be related to cancers and abnormal growths in the intestinal tract. So, COX inhibitors have been shown to reduce the occurrence of cancers and pre-cancerous growths, as well as neurodegenerative diseases.

8. The composition of claim 1, wherein the use of the TMA-PHT complex, or simply its TMA component or other of its components is made as a preventive medical element (s) in an amount effective for a significant prevention rate for any specific disease, including Covid19 viral disease, (also taking advantage of the very low risk of rejection by the patient due to the low-zero toxicity of this OLIVEVIR's TMA-PHT complex.

9. The composition of claim 1, wherein TMA and/or PHT or other of its components are used as a direct or indirect treatment for any infection, including Covid19, and including the use of TMA and/or PHT in an amount effective for the prevention or treatment of inflammation, especially critical during cytokine storm episodes in humans. Taking into account that every place where any viral protein like the SARS-CoV-2 viral ones interacts with a human protein it represents a potential pharmacological site, therefore the various elements of this TMA-PHT drug cocktail could act on different objectives.

10. The composition of claim 1, wherein the use of TMA-PHT is for the treatment and/or prevention of thrombosis.

11. The composition of claim 1, wherein the use of TMA-PHT—or any of its components as an antibiotic drug for any infections including COVID19 and/or antiproliferative and/or anti-inflammatory drug to obtain a reduction in the generation of pro-inflammatory cytokines in any disease including COVID19, as it has been proven in our trials to inhibit the spread of the HIV virus by inhibiting replication of a primary HIV-1 isolated as well as decreased cytopathic effect and p24 antigen levels in MT2 cells and attenuated intracellular oxidative stress by inhibiting the production of NO and H2O2.

12. The composition of claim 1, wherein the use of TMA-PHT—or any of its components like MA—as an immuno-modulator and to improve endothelial function, avoiding lipid peroxidation and suppressing the generation of superoxide anions, taking advantage of the fact that these penta-cyclic Triterpenes of this invention possess antioxidant properties, and Maslinic acid in particular exhibits properties pro and anti-inflammatory depending on its chemical structure and its dose, and may be useful to modulate the immune response due to the immune-modulatory behavior of this triterpene.

13. The composition of claim 1, wherein the use of the TMA-PHT complex, or any of its components, in any type of pharmaceutical form, in any Galenic administration form and at any dose per individual and day, and the process for preventing or treating a disease or medical condition in a subject in need thereof comprising: administering to said subject a composition comprising purified amounts of TMA, or said purified amounts mixed with PHT; wherein said TMA compound comprises 20%-80% of the dry weight of the composition. This claim includes its use for prevention of COVID 19, according to claim n° 8, as far as TMA-PHT complex may enhance the action of the antibodies against the spike protein (generated by vaccination or adoptive transfer) to prevent the severe acute respiratory syndrome coronavirus 2 (SARS-CoV-2) from interacting with the receptor for the converting enzyme of angiotensin 2 (ACE2) in host cells. This claim also includes the use of TMA-PHT complex combined with other phytochemical supplement not obtained from Olive trees, including the ones composed of extracts from three plants ingredients called RWS: Rosmarinus officinalis, Withania somnifera and Sophora japonica, with specific levels of carnosol/carnosic acid, with aferin A and luteolin, respectively. The synergies obtained from each ingredient TMA-PHT and RWS contributes to an important activation of the Nrf2 pathway in unique ways, leading to the up-regulation of cyto-protective human genes and a maximum protection of cells against oxidative stress.

14. A method of using TMA-PHT complex, and/or any of its components or combinations according to claim 1, as a Nutraceutical product or food supplement for humans or animals.

15. A method for obtaining and preparing TMA and PHT as set forth in claim 1, consisting primarily of a mixture of a physiologically acceptable mixture of TMA and PHT phytochemicals, all of which include the steps of the extraction of TMA from fruits, seeds and leaves of olive, and/or products obtained in a manufacturing process of olive oil, or mixtures thereof, with water, a hydrophilic organic solvent or mixtures thereof, and then concentrating the resulting TMA extract on one hand and in the other hand: a method for obtaining and preparing PHT consisting of a mixture of polyphenols, mainly Hydroxytyrosol and Oleuropein in a physiologically acceptable mixture thereof, all of which includes the steps of their extraction from plants of olive, or products obtained in a manufacturing process of olive oil, or mixtures thereof, with water, an organic solvent or mixtures thereof, and then concentrating the resulting extract, and then carrying out a purification treatment by fractionation of the resulting extract.

16. The method of claim 15, further optionally comprising the step of treating the resulting extracted substance or liquid olive extract with a basic medium, a basic substance, or both, and the method of claim 15 optionally further comprising the step of: treating the resulting extracted substance or the extracted liquid with an acidic medium, an acidic substance, or both, as well as the method of claim 15 wherein the TMA and PHT products obtained in the olive oil manufacturing process from the olive plant (fruits, seeds, bark, leaves, stems, and olive sprouts or germs) and dry products, powdered products and defatted products of these ingredients, comprise at least one member selected from the group consisting of compressed residues, extraction residues, squeezed oil, extracted oil, foam of degummed oil, de-acidified foam oil, dark oil, agent waste bleach, deodorized foam, juice expressed in oil, sewage, and waste filter media.

17. The method of claim 15, wherein the residues of the olive oil production process result in phytochemicals, the extraction of which is carried out using water, a hydrophilic organic solvent or mixtures thereof or where the extraction is carried out under pressure at which the precipitates formed by cooling are recovered and at which the total content of oleanolic acid, maslinic acid and physiologically acceptable products thereof, present in a mixture comprising oleanolic and maslinic acid prepared according to the fractionation treatment —purification consisting of recrystallization, re-precipitation, purification by normal phase chromatography, purification by reverse phase chromatography, purification by normal phase and reverse phase chromatography, decolorization and deodorization.

18. The method of claim 14, comprising the step of administering a composition to humans or animals, the composition comprising one or more phytochemicals selected from the group of Olive Terpenes (TMA) and/or Polyphenols (PHT) and combinations thereof and that may include phytochemicals from other plants to reinforce the amounts of very minor phytochemicals in the olive, including carnosol, carnosic acid, shogaol, gingerol, luteolin and/or conferin A in an amount effective to activate the Nrf2 pathway (NF-E2 related factor 2), the composition comprises at least one other phytochemical ingredient selected from the group consisting of rosemary, ginger, ashwagandha, milk thistle and bacopa. Also rosemary extract (ursolic acid), ginger extract in addition to milk thistle extract and Bacopa monnieri extract, being approximately the ratio between said rosemary extract, ginger extract, luteolin, milk thistle extract and Bacopa monnieri extract.

19. The method of claim 15, wherein the TMA-PHT complex or the TMA compounds are made more soluble and bioavailable using one or any combination of the described methods including the preparation in cyclo-dextrines (CD's) or inclusion complexes to increase the solubility and bioavailability of the Triterpenic complex TMA.

20. The method of claim 14, including use of the phytochemical complex TMA-PHT (OLIVEVIR as preventive of COVID19, as well as its usage during the three phases of COVID19 disease with its different therapeutical targets as described in this patent and also its use in post-COVID stage treatments, when required, including PPCS (persistent post-COVID-19 syndrome) cases.
